(19) United States
(12) Patent Application Publication
(10) Pub. No.: US 2025/0270583 A1
(43) Pub. Date: Aug. 28, 2025

(54) Methods for Suppression of Gene Silencing in Plants

(71) Applicant: BioDrive, Inc., Alameda, CA (US)

(72) Inventor: Cory Henderson, Alameda, CA (US)

(21) Appl. No.: 19/123,383

(22) PCT Filed: Oct. 25, 2023
(86) PCT No.: PCT/US2023/035870

§ 371 Date: Apr. 22, 2025

Related U.S. Application Data
(60) Provisional application No. 63/419,692, filed on Oct. 26, 2022.
(60) Provisional application No. 63/421,560, filed on Nov. 1, 2022.
(60) Provisional application No. 63/510,319, filed on Jun. 26, 2023.

(51) Int. Cl. C12N 15/82; C07K 14/325; C12N 9/10; C12N 9/12; C12N 9/22; C12N 15/11; C12N 15/113

(52) U.S. Cl. CPC C12N 15/8286; C07K 14/325; C12N 9/1007; C12N 9/1247; C12N 9/127; C12N 9/22; C12N 9/222; C12N 15/11; C12N 15/113; C12Y 207/07006; C12Y 207/07048; C12Y 301/26003; C12N 2310/14; C12N 2310/20

(57) ABSTRACT

The present disclosure relates to compositions and methods for the suppression of silencing of a gene of interest in plants. Also disclosed are vectors, transgenic plants and plant parts comprising genetic constructs for suppressing endogenous plant silencing of a gene of interest.

### FIELD OF THE DISCLOSURE

The present disclosure relates to compositions and methods for the suppression of gene silencing in plants, as well as plants comprising anti-silencing compositions.

### BACKGROUND

Genetically modified plants are a staple of modern agriculture. Genetic modifications allow for improved crop traits, such as pest resistance, hardiness, productivity, and nutrient content, among many other attributes. Unfortunately, many genetically modified plants suffer from the silencing of transgenes, suppressing the desired traits.

The major components of RNA silencing include both transitive and systemic small RNAs, known as secondary sRNAs. Double-stranded RNAs trigger systemic silencing pathways to negatively regulate gene expression. The secondary siRNAs generated as a result of transitive silencing play a significant role in gene silencing, especially in antiviral defense. RNA silencing is a sequence-specific RNA degradation and inactivation mechanism, operative in most eukaryotes. It has also been implicated in the epigenetic events resulting in suppression of repetitive sequences including transposable elements (TEs) and imprinted genes. RNA silencing is now used as an umbrella term to encompass suppression of gene expression by all kinds of 21 to 24-nucleotide (nt) small RNAs (sRNAs), generated primarily due to the activity of enzymes like Dicers or Dicer-like proteins (DCLs).

sRNAs can be broadly categorized as small interfering RNAs (siRNAs) and microRNAs (miRNAs) in plant systems. They are grouped based on the mechanism of their biogenesis from precursor double-stranded RNAs (dsRNAs) or hairpin RNAs, respectively. Functional sRNAs are incorporated in the argonaute (AGO) protein of the RNA-induced silencing complex (RISC) that can act as a site-specific endonuclease on the cytoplasmic transcripts to enable posttranscriptional gene silencing (PTGS), sRNAs can also target the genomic DNA in the nucleus to facilitate transcriptional gene silencing (TGS), siRNAs can target endogenous sequences as well as exogenous sequences such as viruses and transgenes serving as the first line of host defense

There is an ongoing and unmet need for compositions and methods to reduce plant siRNA-mediated gene silencing.

### BRIEF SUMMARY

In one aspect, the present disclosure provides a genetic construct comprising: a plant host factor sequence operably linked to a genetic cargo sequence comprising a gene of interest; and an siRNA sequence, wherein the siRNA sequence increases the likelihood that silencing of the genetic construct will lead to silencing of the plant host factor sequence.

In one aspect, the present disclosure provides a transgenic plant, plant part, or plant cell comprising a genetic construct of the disclosure.

In one aspect, the present disclosure provides a vector comprising a construct of the disclosure.

In one aspect, the present disclosure provides a transgenic plant, or part thereof, comprising a genetic construct comprising: a plant host factor sequence operably linked to a genetic cargo sequence comprising a gene of interest; and an siRNA sequence, wherein the siRNA sequence increases the likelihood that silencing of the genetic construct will lead to silencing of the plant host factor sequence.

In one aspect, the present disclosure provides a method of delivering a genetic construct according to an embodiment herein.

In one aspect, the present disclosure provides a method of suppressing endogenous silencing of a gene of interest in a plant, plant part, or plant cell, comprising: transforming the plant, plant part, or plant cell with a genetic construct comprising: (i) a plant host factor sequence operably linked to a genetic cargo sequence comprising a gene of interest; and (ii) an siRNA sequence, wherein the siRNA sequence increases the likelihood that silencing of the genetic construct will lead to silencing of the plant host factor sequence. wherein expression of the genetic construct suppresses endogenous silencing of the gene of interest.

In one aspect, the present disclosure provides a method of increasing expression of a gene of interest in a plant, plant part, or plant cell, comprising: transforming the plant, plant part, or plant cell with a genetic construct comprising: (i) a plant host factor sequence operably linked to a genetic cargo sequence comprising a gene of interest; and (ii) an siRNA sequence, wherein the siRNA sequence increases the likelihood that silencing of the genetic construct will lead to silencing of the plant host factor sequence, wherein expression of the genetic construct suppresses endogenous silencing of the gene of interest.

### BRIEF DESCRIPTION OF THE SEVERAL VIEWS OF THE DRAWINGS

For a more complete understanding of the features and advantages of the present invention, reference is now made to the detailed description of the invention along with the accompanying figures and in which:

FIG. 1A-1C show illustrative genetic constructs of the disclosure. FIG. 1A shows a genetic construct comprising a host factor sequence in proximity to a genetic cargo, wherein the host factor sequence comprises siRNA sequences. FIG. 1B shows a construct comprising a host factor sequence and a genetic cargo operating under the same promoter. FIG. 1C shows a construct comprising a host factor sequence and a genetic cargo operating under a separate promoter.

FIG. 2 shows an illustrative retrotransposon-based vector of the disclosure.

FIG. 3A-3B show illustrative CRISPR-based vectors of the disclosure. FIG. 3A shows a genetic construct with homology arms that are not necessarily complementary to the host factor sequence. FIG. 3B shows a genetic construct in which one of the homology arms is homologous to a host factor.

FIG. 4A-4C show illustrative vectors of the disclosure comprising a Bt toxin gene and an AGO host factor sequence. FIG. 4A shows a retrotransposon-based vector; FIG. 4B shows a CRISPR-based vector; and FIG. 4C shows a geminivirus comprising a CRISPR-based system.

FIG. 5A-5B show illustrative vectors of the disclosure comprising an ALDH gene and a DCL host factor sequence. FIG. 5A shows a retrotransposon-based vector; and FIG. 5B shows a CRISPR-based vector.

FIG. 6 shows an illustrative genetic construct comprising a host factor sequence and a genetic cargo in proximity.

FIG. 7 shows an illustrative genetic construct comprising a retrotransposon-based system—5′ and 3′ UTR without siRNA sequences.

FIG. 8 shows an illustrative genetic construct comprising a retrotransposon-based system—5′ and 3′ UTR with siRNA sequences.

FIG. 9A-9B show diagrams of processing plants for peptide extraction. FIG. 9A shows extraction at low purities. FIG. 79B shows extraction at high purities.

### DETAILED DESCRIPTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

### Definitions

The term “a” or “an” refers to one or more of that entity, i.e. can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein, “adaptability” is used to refer to a plant's characteristic of being able to grow well in different growing conditions (climate, soils, etc.).

As used herein, a “biomolecule” refers to any molecule produced within a host plant cell. In some embodiments, a biomolecule is a lipid, carbohydrate, nucleic acid, or protein. In some embodiments, a biomolecule is a recombinant protein, e.g., a vaccine antigen, antibody, or antibody fragment.

In the context of the present disclosure a “collection of seeds” is a grouping of seeds mainly containing similar kind of seeds, for example inbred line seeds of the disclosure, but that may also contain, mixed together with another different kind of seeds, for example parental line seeds or hybrid seeds.

As used herein, “event” refers to the unique DNA recombination event that took place in one plant cell, which was then used to generate entire transgenic plants. Plant cells are transformed with a vector carrying a DNA insert of interest. Transformed cells are regenerated into transgenic plants, and each resulting transgenic plant represents a unique event. Different events possess differences in the number of copies of DNA insert in the cell genome, the arrangement of the DNA insert copies and/or the DNA insert location in the genome.

As used herein, a “gene of interest” refers to any gene for which expression, or increased expression, is desired within a host plant cell. In some embodiments, a gene of interest is a transgene. In some embodiments, a gene of interest is an endogenous gene. In some embodiments, a gene of interest is transgenic, cisgenic, or intragenic. In some embodiments, a gene of interest is synthetic.

As used herein, a “genetic cargo” is a polynucleotide comprising a gene of interest. In some embodiments, the genetic cargo is the gene of interest. In some embodiments, the genetic cargo comprises sequences in addition to the gene of interest. In some embodiments, the genetic cargo comprises a promoter. In some embodiments, the genetic cargo comprises upstream or downstream sequences that modify the expression of the gene of interest. In some embodiments, the genetic cargo comprises genes in addition to the gene of interest.

Genetic rearrangement refers to the re-association of genetic elements that can occur spontaneously in vivo as well as in vitro which introduce a new organization of genetic material. For instance, the splicing together of polynucleotides at different chromosomal loci, can occur spontaneously in vivo during both plant development and sexual recombination. Accordingly, recombination of genetic elements by non-natural genetic modification techniques in vitro is akin to recombination events that also can occur through sexual recombination in vivo.

As used herein, a “host factor” refers to a polynucleotide, especially a gene, endogenous to a host plant cell. In some embodiments, the host factor is a gene involved in gene silencing. In some embodiments, the host factor is a gene involved in plant survival. In some embodiments, the host factor is a gene involved in plant fertility.

As used herein, a “host factor sequence” refers to a sequence that is homologous to a host factor. In some embodiments, the host factor sequence is a host factor gene, a part thereof, or a sequence homologous thereto.

As used herein, a “host plant” refers to a plant comprising or intended to comprise a genetic construct as disclosed herein. In some embodiments, the host plant is a monocot. In some embodiments. the host plant is a dicot. In some embodiments, the host plant is an agricultural crop, an agronomical crop, a horticultural crop, or an ornamental plant.

A plant which is not subject to attack or infection by a specific disease or insect is considered “immune” to that disease or insect.

As used herein, “intermediate resistance” refers to a plant that restricts the growth and development of a specific disease or insect, but may exhibit a greater range of symptoms or damage compared to resistant plants. Intermediate resistant plants will usually show less severe symptoms or damage than susceptible plant varieties when grown under similar environmental conditions and/or specific disease(s) and or insect(s) pressure, but may have heavy damage under heavy pressure. Intermediate resistant plants are not immune to the disease(s) and or insect(s).

As used herein, the term “new breeding techniques” or “NBTs” refers to various new technologies developed and/or used to create new characteristics in plants through genetic variation, the aim being targeted mutagenesis, that is targeted introduction of new genes or gene silencing. The following breeding techniques are within the scope of NBTs; targeted sequence changes facilitated through the use of Zinc finger nuclease (ZFN) technology (ZFN-1, ZFN-2 and ZFN-3, see U.S. Pat. No. 9,145,565, incorporated by reference in its entirety). Oligonucleotide directed mutagenesis (ODM, a.k.a., site-directed mutagenesis), Cisgenesis and intragenesis, epigenetic approaches such as RNA-dependent DNA methylation (RdDM, which does not necessarily change nucleotide sequence but can change the biological activity of the sequence), Grafting (on GM rootstock), Reverse breeding, retrotransposon based systems, Agro-infiltration for transient gene expression (agro-infiltration “sensu stricto”, agro-inoculation, floral dip), genome editing with endonucleases such as chemical nucleases, meganucleases, ZFNs, and Transcription Activator-Like Effector Nucleases (TALENs, see U.S. Pat. Nos. 8,586,363 and 9,181,535, incorporated by reference in their entireties), the CRISPR/Cas system (using such as Cas9, Cas12a/Cpf1, Cas13/C2c2, CasX and CasY; also see U.S. Pat. Nos. 8,697,359; 8,771,945; 8,795,965; 8,865,406; 8,871.445; 8,889,356; 8,895,308; 8,906,616; 8,932,814; 8,945,839; 8,993,233; and 8,999,641, which are all hereby incorporated by reference), engineered meganuclease, engineered homing endonucleases, DNA guided genome editing (Gao et al., Nature Biotechnology (2016), doi: 10.1038/nbt.3547, incorporated by reference in its entirety), and Synthetic genomics. A complete description of each of these techniques can be found in the report made by the Joint Research Center (JRC) Institute for Prospective Technological Studies of the European Commission in 2011 and titled “New plant breeding techniques—State-of-the-art and prospects for commercial development”, which is incorporated by reference in its entirety.

Operably linked, Combining two or more molecules in such a fashion that in combination they function properly in a plant cell. For instance, a promoter is operably linked to a structural gene when the promoter controls transcription of the structural gene.

A plant having good “plant adaptability” refers to a plant that will perform well in different growing conditions and seasons.

As used herein, the term “plant cell” includes plant cells whether isolated, in tissue culture or incorporated in a plant or plant part.

As used herein, the term “plant part”, “part thereof” or “parts thereof” includes plant cells, plant protoplasts, plant cell tissue cultures from which plants can be regenerated, plant calli, plant clumps and plant cells that are intact in plants or parts of plants, such as embryos, pollens, ovules, flowers, seeds, heads, rootstocks, scions, stems, roots, anthers, pistils, root tips, leaves, meristematic cells, axillary buds, hypocotyls, cotyledons, ovaries, seed coats, endosperms and the like. In some embodiments, the plant part comprises at least one cell of said plant. In some embodiments, the plant part is further defined as a pollen, a meristem, a cell or an ovule. In some embodiments, a plant regenerated from the plant part has all of the phenotypic and morphological characteristics of a plant of the present disclosure.

As used herein, “regeneration” refers to the development of a plant from tissue culture.

As used herein, “resistance to disease(s) and or insect(s)” refers to a plant that restricts the growth and development of specific disease(s) and or insect(s) under normal disease(s) and or insect(s) attack pressure when compared to susceptible plants. These plants can exhibit some symptoms or damage under heavy disease(s) and or insect(s) pressure. Resistant plants are not immune to the disease(s) and or insect(s).

As used herein, the term “sequence identity” refers to the extent to which two optimally aligned polynucleotides or polypeptide sequences are invariant throughout a window of alignment of residues, e.g. nucleotides or amino acids. An “identity fraction” for aligned segments of a test sequence and a reference sequence is the number of identical residues which are shared by the two aligned sequences divided by the total number of residues in the reference sequence segment, i.e. the entire reference sequence or a smaller defined part of the reference sequence. “Percent identity” is the identity fraction times 100. Comparison of sequences to determine percent identity can be accomplished by a number of well-known methods, including for example by using mathematical algorithms, such as, for example, those in the BLAST suite of sequence analysis programs. Unless noted otherwise, the term “sequence identity” in the claims refers to sequence identity as calculated by Clustal Omega R using default parameters.

As used herein, “single locus converted (conversion) plants” refer to plants which are developed by a plant breeding technique called backcrossing, wherein essentially all of the desired morphological and physiological characteristics of a plant are recovered in addition to a single locus transferred into the plant via the backcrossing technique or via genetic engineering. A single locus converted plant can also be referred to a plant with a single locus conversion obtained though simultaneous and/or artificially induced mutagenesis or through the use of New Breeding Techniques described in the present disclosure. In some embodiments, the single locus converted plant has otherwise all of the desired morphological and physiological characteristics of the original variety in addition to a single locus converted by spontaneous and/or artificially induced mutations, which is introduced and/or transferred into the plant by the plant breeding techniques such as backcrossing. In other embodiments, the single locus converted plant has otherwise all of the desired morphological and physiological characteristics of the original variety in addition to a single locus, gene or nucleotide sequence(s) converted, mutated, modified or engineered through the New Breeding Techniques taught herein. In the present disclosure, single locus converted (conversion) can be interchangeably referred to single gene converted (conversion).

As used herein, “siRNA” or “Small interfering RNA” refers to a class of double-stranded RNA molecules, typically 20-24 (normally 21) base pairs in length, that operate within the RNA interference (RNAi) pathway.

A plant that is “susceptible to disease(s) and or insect(s)” is defined as a plant that has the inability to restrict the growth and development of specific disease(s) and or insect(s). Plants that are susceptible will show damage when infected and are more likely to have heavy damage under moderate levels of specific disease(s) and or insect(s).

Transgenic plant. A genetically modified plant which contains at least one transgene.

A plant that is “tolerant to abiotic stress” has the ability to endure abiotic stress without serious consequences for growth, appearance and yield.

Transformation of plant cells refers to a process by which DNA is integrated into the genome of a plant cell. The integration may be transient or stable. “Stably” refers to the permanent, or non-transient retention and/or expression of a polynucleotide in and by a cell genome. Thus, a stably integrated polynucleotide is one that is a fixture within a transformed cell genome and can be replicated and propagated through successive progeny of the cell or resultant transformed plant. Transformation may occur under natural or artificial conditions using various methods well known in the art. Transformation may rely on any known method for the insertion of nucleic acid sequences into a prokaryotic or eukaryotic host cell, including Agrobacterium-mediated transformation protocols, viral infection, whiskers, electroporation, heat shock, lipofection, polyethylene glycol treatment, micro-injection, and particle bombardment.

As used herein, “uniformity.” describes the similarity between plants or plant characteristics which can be a described by qualitative or quantitative measurements.

A plant “variety” as used by one skilled in the art of plant breeding means a plant grouping within a single botanical taxon of the lowest known rank which can be defined by the expression of the characteristics resulting from a given genotype or combination of phenotypes, distinguished from any other plant grouping by the expression of at least one of the said characteristics and considered as a unit with regard to its suitability for being propagated unchanged (International convention for the protection of new varieties of plants). The term “variety” can be interchangeably used with “cultivar” or “line”.

### Compositions and Methods for Reducing Sirna-Mediated Gene Silencing in Plants

The present disclosure relates to the use of siRNAs and siRNA pathway genes to prevent silencing of genetic constructs, e.g., transgenic constructs in stable transgenic lines, and to allow for sustained mobilization of genes of interest, e.g., transgenes, in plant populations. In contrast to existing technologies. the novel compositions and methods of the present disclosure are aimed not at expression of dsRNA to drive an RNAi based response in the plant or in insect pests, but instead to make use of the RNAi response in the plant to prevent effective silencing of genes of interest, e.g., transgenes.

### Genetic Constructs

Disclosed herein are genetic constructs comprising a genetic cargo comprising a gene of interest, a host factor sequence, and an siRNA sequence.

### Gene of Interest

Genetic constructs of the disclosure comprise a genetic cargo comprising a gene of interest. The gene of interest is a gene intended for expression within the host plant cell. In some embodiments, the gene of interest is transgenic, intragenic, or cisgenic. In some embodiments, the gene of interest is a transgene. In some embodiments, the gene of interest is a gene endogenous to the host plant cell. In some embodiments, the gene of interest is a gene from a related plant species. In some embodiments, the gene of interest is a synthetic gene.

In some embodiments, the methods used herein allow the gene of interest to be mobilized. In some embodiments, the methods used herein allow the gene of interest to be replicated within the plant genome.

In some embodiments, the gene of interest is involved in pest resistance. In some embodiments, the gene of interest is a biocidal gene. In some embodiments, the gene of interest is a viricidal, bactericidal, insecticidal, or fungicidal gene.

In some embodiments, the gene of interest is involved in improving a plant trait.

In some embodiments, the gene of interest is involved in production of a biomolecule. In some embodiments, the biomolecule is a lipid, protein, carbohydrate, or nucleic acid.

### Herbicide Resistance

In some embodiments, the gene of interest is related to herbicide resistance. Numerous herbicide resistance genes are known and may be employed with the invention. A non-limiting example is a gene conferring resistance to a herbicide that inhibits the growing point or meristem such as imidazolinone or sulfonylurea herbicides. For example, by Lee et al., EMBO J., 7:1241, 1988; Gleen et al., Plant Molec. Biology. 18:1185, 1992; and Miki et al., Theor. Appl. Genet., 80:449, 1990. As a non-limiting example, a gene may be employed to confer resistance to the exemplary sulfonylurea herbicide nicosulfuron.

Resistance genes for glyphosate (resistance conferred by mutant 5-enolpyruvylshikimate-3-phosphate synthase (EPSPS) and aroA genes, respectively) and other phosphono compounds such as glufosinate (phosphinothricin acetyltransferase (PAT) and Streptomyces hygroscopicus phosphinothricin acetyltransferase (bar) genes) may also be used. See, for example, U.S. Pat. No. 4,940,835 to Shah et al., which discloses the nucleotide sequence of a form of EPSPS that can confer glyphosate resistance. Non-limiting examples of EPSPS transformation events conferring glyphosate resistance are provided by U.S. Pat. Nos. 6,040,497 and 7,632,985. The MON89788 event disclosed in U.S. Pat. No. 7,632,985 in particular is beneficial in conferring glyphosate tolerance in combination with an increase in average yield relative to prior events.

A DNA molecule encoding a mutant aroA gene can be obtained under ATCC Accession No. 39256, and the nucleotide sequence of the mutant gene is disclosed in U.S. Pat. No. 4,769,061 to Comai. A hygromycin B phosphotransferase gene from E. coli that confers resistance to glyphosate in tobacco callus and plants is described in Penaloza-Vazquez et al., Plant Cell Reports. 14:482, 1995. European Patent Application Publication No. EP0333033 to Kumada et al., and U.S. Pat. No. 4,975,374 to Goodman et al., disclose nucleotide sequences of glutamine synthetase genes that confer resistance to herbicides such as L-phosphinothricin. The nucleotide sequence of a phosphinothricin acetyltransferase gene is provided in European Patent Application Publication No. EP0242246 to Leemans et al. DeGreef et al. (Biotechnology; 7:61, 1989) describe the production of transgenic plants that express chimeric bar genes coding for phosphinothricin acetyl transferase activity. Exemplary genes conferring resistance to a phenoxy class herbicide haloxyfop and a cyclohexanedione class herbicide sethoxydim are the Acct-S1, Acct-S2 and Acct-S3 genes described by Marshall et al., (Theor. Appl. Genet., 83:435, 1992). As a non-limiting example, a gene may confer resistance to other exemplary phenoxy class herbicides that include, but are not limited to, quizalofop-p-ethyl and 2,4-dichlorophenoxyacetic acid (2,4-D).

Genes are also known that confer resistance to herbicides that inhibit photosynthesis such as, for example, triazine herbicides (psbA and gs+ genes) and benzonitrile herbicides (nitrilase gene). As a non-limiting example, a gene may confer resistance to the exemplary benzonitrile herbicide bromoxynil. Przibila et al. (Plant Cell, 3:169, 1991) describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441, and 67442. Cloning and expression of DNA coding for a glutathione S-transferase is described by Hayes et al. (Biochem. J., 285:173, 1992). 4-hydroxyphenylpyruvate dioxygenase (HPPD) is a target of the HPPD-inhibiting herbicides, which deplete plant plastoquinone and vitamin E pools. Rippert et al. (Plant Physiol., 134:92, 2004) describes an HPPD-inhibitor resistant tobacco plant that was transformed with a yeast-derived prephenate dehydrogenase (PDH) gene. Protoporphyrinogen oxidase (PPO) is the target of the PPO-inhibitor class of herbicides; a PPO-inhibitor resistant PPO gene was recently identified in Amaranthus tuberculatus (Patzoldt et al., PNAS, 103(33): 12329, 2006). The herbicide methyl viologen inhibits CO₂ assimilation. Foyer et al. (Plant Physiol., 109:1047, 1995) describe a plant overexpressing glutathione reductase (GR) that is resistant to methyl viologen treatment.

Siminszky (Phytochemistry Reviews, 5:445, 2006) describes plant cytochrome P450-mediated detoxification of multiple, chemically unrelated classes of herbicides. Modified bacterial genes have been successfully demonstrated to confer resistance to atrazine, a herbicide that binds to the plastoquinone-binding membrane protein Q_{B} in photosystem II to inhibit electron transport. See, for example, studies by Cheung et al. (PNAS, 85:391, 1988), describing tobacco plants expressing the chloroplast psbA gene from an atrazine-resistant biotype of Amaranthus hybridus fused to the regulatory sequences of a nuclear gene, and Wang et al. (Plant Biotech. J., 3:475, 2005), describing transgenic alfalfa, Arabidopsis, and tobacco plants expressing the atzA gene from Pseudomonas sp. that were able to detoxify atrazine.

Bayley et al. (Theor. Appl. Genet., 83:645, 1992) describe the creation of 2,4-D-resistant transgenic tobacco and cotton plants using the 2,4-D monooxygenase gene tfdA from Alcaligenes eutrophus plasmid pJP5. U.S. Patent Application Publication No. 20030135879 describes the isolation of a gene for dicamba monooxygenase (DMO) from Psueodmonas maltophilia that is involved in the conversion of dicamba to a non-toxic 3,6-dichlorosalicylic acid and thus may be used for producing plants tolerant to this herbicide.

Other examples of herbicide resistance have been described, for instance, in U.S. Pat. Nos. 6,803,501; 6,448,476; 6,248,876; 6,225,114; 6,107,549; 5,866,775; 5,804,425; 5,633,435; 5,463,175.

### Disease and Pest Resistance

In some embodiments, the gene of interest is related to disease and/or pest resistance. Plant defenses are often activated by specific interaction between the product of a disease resistance gene (R) in the plant and the product of a corresponding avirulence (Avr) gene in the pathogen. A plant line can be transformed with a cloned resistance gene to engineer plants that are resistant to specific pathogen strains. See, for example Jones et al. (Science, 266:789-793, 1994) (cloning of the tomato Cf-9 gene for resistance to Cladosporium flavum); Martin et al. (Science, 262:1432-1436, 1993) (tomato Pto gene for resistance to Pseudomonas syringae pv. tomato); and Mindrinos et al. (Cell, 78(6):1089-1099, 1994) (Arabidopsis RPS2 gene for resistance to Pseudomonas syringae).

A viral-invasive protein or a complex toxin derived therefrom may also be used for viral disease resistance. For example, the accumulation of viral coat proteins in transformed plant cells imparts resistance to viral infection and/or disease development effected by the virus from which the coat protein gene is derived and related viruses. See Beachy et al. (Ann. Rev. Phytopathol., 28:451, 1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus, and tobacco mosaic virus.

A virus-specific antibody may also be used. See, for example, Tavladoraki et al. (Nature, 366:469-472, 1993), who show that transgenic plants expressing recombinant antibody genes are protected from virus attack. Virus resistance has also been described in, for example, U.S. Pat. Nos. 6,617,496; 6,608,241; 6,015,940; 6,013,864; 5,850,023 and 5,304,730. Additional means of inducing whole-plant resistance to a pathogen include modulation of the systemic acquired resistance (SAR) or pathogenesis related (PR) genes, for example genes homologous to the Arabidopsis thaliana NIM1/NPR1/SAI1, and/or by increasing salicylic acid production (Ryals et al., Plant Cell, 8:1809-1819, 1996).

Logemann et al. (Biotechnology, 10:305-308, 1992), for example, disclose transgenic plants expressing a barley ribosome-inactivating gene that have an increased resistance to fungal disease. Plant defensins may be used to provide resistance to fungal pathogens (Thomma et al., Planta, 216:193-202, 2002). Other examples of fungal disease resistance are provided in U.S. Pat. Nos. 6,653,280; 6,573,361; 6,506,962; 6,316,407; 6,215,048; 5,516,671; 5,773,696; 6,121,436; and 6,316,407.

Nematode resistance has been described in, for example, U.S. Pat. No. 6,228,992, and bacterial disease resistance has been described in, for example, U.S. Pat. No. 5,516,671.

The use of the herbicide glyphosate for disease control in hemp plants containing event MON89788, which confers glyphosate tolerance, has also been described in U.S. Pat. No. 7,608,761.

### Insect Resistance

In some embodiments, the gene of interest is related to insect resistance.

One example of an insect resistance gene includes a Bacillus thuringiensis protein, a derivative thereof, or a synthetic polypeptide modeled thereon. See, for example, Geiser et al. (Gene, 48(1):109-118, 1986), who disclose the cloning and nucleotide sequence of a Bacillus thuringiensis δ-endotoxin gene. Moreover, DNA molecules encoding δ-endotoxin genes can be purchased from the American Type Culture Collection, Manassas, Va., for example, under ATCC Accession Nos. 40098, 67136, 31995 and 31998. Another example is a lectin. See, for example, Van Damme et al., (Plant Molec. Biol., 24:825-830, 1994), who disclose the nucleotide sequences of several Clivia miniata mannose-binding lectin genes. A vitamin-binding protein may also be used, such as, for example, avidin. See PCT Application No. US93/06487, the contents of which are hereby incorporated by reference. This application teaches the use of avidin and avidin homologues as larvicides against insect pests.

In some embodiments, the gene of interest encodes a pesticidal protein. In some embodiments, the pesticidal protein is from Bacillus thuringiensis. In some embodiments, the gene encodes a Vip or Cry protein.

Yet another insect resistance gene is an enzyme inhibitor, for example, protease, proteinase, or amylase inhibitors. See, for example, Abe et al. (J. Biol. Chem., 262:16793-16797, 1987) describing the nucleotide sequence of a rice cysteine proteinase inhibitor; Linthorst et al. (Plant Molec. Biol., 21:985-992. 1993) describing the nucleotide sequence of a cDNA encoding tobacco proteinase inhibitor I; and Sumitani et al. (Biosci. Biotech. Biochem., 57:1243-1248, 1993) describing the nucleotide sequence of a Streptomyces nitrosporeus α-amylase inhibitor.

An insect-specific hormone or pheromone may also be used. See, for example, the disclosure by Hammock et al. (Nature, 344:458-461, 1990) of baculovirus expression of cloned juvenile hormone esterase, an inactivator of juvenile hormone; Gade and Goldsworthy (Eds. Physiological System in Insects, Elsevier Academic Press, Burlington, Mass., 2007), describing allostatins and their potential use in pest control; and Palli et al. (Vitam. Horm., 73:59-100, 2005), disclosing use of ecdysteroid and ecdysteroid receptor in agriculture. The diuretic hormone receptor (DHR) was identified in Price et al. (Insect Mol. Biol., 13:469-480, 2004) as another potential candidate target of insecticides.

Still other examples include an insect-specific antibody or an immunotoxin derived therefrom and a developmental-arrestive protein. See Taylor et al. (Seventh Int'l Symposium on Molecular Plant-Microbe Interactions, Edinburgh, Scotland, Abstract W97, 1994), who described enzymatic inactivation in transgenic tobacco via production of single-chain antibody fragments. Numerous other examples of insect resistance have been described. See, for example, U.S. Pat. Nos. 6,809,078; 6,713,063; 6,686,452; 6,657,046; 6,645,497; 6,642,030; 6,639,054; 6,620,988; 6,593,293; 6,555,655; 6,538,109; 6,537,756; 6,521,442; 6,501,009; 6,468,523; 6,326,351; 6,313,378; 6,284,949; 6,281,016; 6,248,536; 6,242,241; 6,221,649; 6,177,615; 6,156,573; 6,153,814; 6,110,464; 6,093,695; 6,063,756; 6,063,597; 6,023,013; 5,959,091; 5,942,664; 5,942,658, 5,880,275; 5,763,245 and 5,763,241.

In some embodiments, the gene of interest is a plant-incorporated protectant, such as any of those listed in Table 1 below.

TABLE 1
Illustrative Plant-Incorporated Protectant Genes of interest

| | Company | Pesticide |
|---|---|---|
| Plant-Incorporated | and Trade | Registration |
| Protectants (PIPs) | Names | Numbers |
| Potato | Potato | |
| Cry3A Potato PC Code 006432 | Naturemark | 524-474 |
| | New Leaf Monsanto | |
| Cry3A & PLRV Potato | Monsanto | 524-498 |
| PC Codes 006432, 006469 | New Leaf Plus | |
| Corn | | |
| Cry1Ab Corn Event 176 | Mycogen Seeds/Dow | 68467-1 |
| PC Code 006458 | Agro | 66736-1 |
| | Syngenta Seeds | |
| Cry1Ab Corn Event Bt11 EPA | Agrisure CB (with | 67979-1 |
| PC Code 006444 OECD Unique, | Yieldgard) | 65268-1 |
| Identifier SYN-BTØ11-1 | Attribute Insect | |
| | Protected Sweet Corn | |
| | Syngenta Seeds | |
| Cry1Ab Corn Event MON 801 | Monsanto | 524-492 |
| Cry1Ab corn Event MON 810 PC | Monsanto | 524-489 |
| Code 006430 OECD Unique | | |
| Identifier MON-ØØ810-6 | | |
| Cry1Ac Corn PC Code 006463 | Dekalb Genetics c/o | 69575-2 |
| | Monsanto | |
| | BT-XTRA | |
| Cry1F corn Event TC1507 PC | Mycogen Seeds/Dow | 68467-2 |
| Code 006481 OECD Unique | Agro | 29964-3 |
| Identifier DAS-Ø1507-1 | Pioneer Hi-Bred/ | |
| | Dupont | |

### Bt Proteins

The most widely used microbial biopesticide is the insect pathogenic bacteria Bacillus thuringiensis (Bt), which produces a protein crystal (the Bt δ-endotoxin) during bacterial spore formation that is capable of causing lysis of gut cells when consumed by susceptible insects. Microbial Bt biopesticides consist of bacterial spores and δ-endotoxin crystals mass-produced in fermentation tanks and formulated as a sprayable product. Bt does not harm vertebrates and is safe to people, beneficial organisms and the environment. Thus, Bt sprays are a growing tactic for pest management on fruit and vegetable crops where their high level of selectivity and safety are considered desirable, and where resistance to synthetic chemical insecticides is a problem. Bt sprays have also been used on commodity crops such as maize, soybean and cotton, but with the advent of genetic modification of plants, farmers are increasingly growing Bt transgenic crop varieties.

As used herein, “transgenic insecticidal trait” refers to a trait exhibited by a plant that has been genetically engineered to express a nucleic acid or polypeptide that is detrimental to one or more pests. In one embodiment, the plants of the present disclosure are resistant to attach and/or infestation from any one or more of the pests of the present disclosure. In one embodiment, the trait comprises the expression of vegetative insecticidal proteins (VIPs) from Bacillus thuringiensis, lectins and proteinase inhibitors from plants, terpenoids, cholesterol oxidases from Streptomyces spp., insect chitinases and fungal chitinolytic enzymes, bacterial insecticidal proteins and early recognition resistance genes. In another embodiment, the trait comprises the expression of a Bacillus thuringiensis protein that is toxic to a pest. In one embodiment, the Bt protein is a Cry protein (crystal protein). Bt crops include Bt corn, Bt cotton and Bt soy. Bt toxins can be from the Cry family (see, for example, Crickmore et al., 1998, Microbiol. Mol. Biol. Rev. 62:807-812), which are particularly effective against Lepidoptera, Coleoptera and Diptera.

Bt Cry and Cyt toxins belong to a class of bacterial toxins known as pore-forming toxins (PFT)

that are secreted as water-soluble proteins undergoing conformational changes in order to insert into, or to translocate across, cell membranes of their host. There are two main groups of PFT: (i) the α-helical toxins, in which α-helix regions form the trans-membrane pore, and (ii) the β-barrel toxins, that insert into the membrane by forming a ≢-barrel composed of βsheet hairpins from each monomer. See, Parker M W, Feil S C, “Porc-forming protein toxins: from structure to function,” Prog. Biophys. Mol. Biol. 2005 May; 88(1):91-142. The first class of PFT includes toxins such as the colicins, exotoxin A, diphtheria toxin and also the Cry three-domain toxins. On the other hand, acrolysin, α-hemolysin, anthrax protective antigen, cholesterol-dependent toxins as the perfringolysin O and the Cyt toxins belong to the β-barrel toxins. Id. In general, PFT producing-bacteria secrete their toxins and these toxins interact with specific receptors located on the host cell surface. In most cases, PFT are activated by host proteases after receptor binding inducing the formation of an oligomeric structure that is insertion competent. Finally, membrane insertion is triggered, in most cases, by a decrease in pH that induces a molten globule state of the protein. Id.

The development of transgenic crops that produce Bt Cry proteins has allowed the substitution of chemical insecticides by environmentally friendly alternatives. In transgenic plants the Cry toxin is produced continuously, protecting the toxin from degradation and making it reachable to chewing and boring insects. Cry protein production in plants has been improved by engineering cry genes with a plant biased codon usage, by removal of putative splicing signal sequences and deletion of the carboxy-terminal region of the protoxin. See, Schuler T H, et al., “Insect-resistant transgenic plants,” Trends Biotechnol. 1998; 16:168-175. The use of insect resistant crops has diminished considerably the use of chemical pesticides in areas where these transgenic crops are planted. See, Qaim M. Zilberman D, “Yield effects of genetically modified crops in developing countries,” Science, 2003 Feb. 7; 299(5608):900-2.

Known Cry proteins include: δ-endotoxins including but not limited to: the Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry28, Cry29, Cry30, Cry31, Cry32, Cry33, Cry34, Cry35, Cry36, Cry37, Cry38, Cry39, Cry40, Cry41, Cry42, Cry43, Cry44, Cry45, Cry46, Cry47, Cry49, Cry51, Cry52, Cry53, Cry54, Cry55, Cry56, Cry57, Cry58, Cry59, Cry60, Cry61, Cry62, Cry63, Cry64, Cry65, Cry66, Cry67, Cry68, Cry69, Cry70 and Cry71 classes of δ-endotoxin genes and the B. thuringiensis cytolytic cyt1 and cyt2 genes.

### Resistance to Abiotic Stress

In some embodiments, the gene of interest is related to mitigating the effects of abiotic stress. Abiotic stress includes dehydration or other osmotic stress, salinity, high or low light intensity, high or low temperatures, submergence, exposure to heavy metals, and oxidative stress. Delta-pyrroline-5-carboxylate synthetase (P5CS) from mothbean has been used to provide protection against general osmotic stress. Mannitol-1-phosphate dehydrogenase (mt1D) from E. coli has been used to provide protection against drought and salinity. Choline oxidase (codA from Arthrobactor globiformis) can protect against cold and salt. E. coli choline dehydrogenase (betA) provides protection against salt. Additional protection from cold can be provided by omega-3-fatty acid desaturase (fad7) from Arabidopsis thaliana. Trehalose-6-phosphate synthase and levan sucrase (SacB) from yeast and Bacillus subtilis, respectively, can provide protection against drought (summarized from Annex II Genetic Engineering for Abiotic Stress Tolerance in Plants, Consultative Group On International Agricultural Research Technical Advisory Committee). Overexpression of superoxide dismutase can be used to protect against superoxides, see U.S. Pat. No. 5,538,878.

### Plant Molecular Farming

Since the advancements of plant genetic engineering in the 1980s, plants have been used for the production of economically valuable, biologically active non-native proteins or biopharmaceuticals, the concept termed as plant molecular farming (PMF). Many biopharmaceuticals including recombinant vaccine antigens, monoclonal antibodies, and other commercially viable proteins are produced in plants, some of which are in the pre-clinical and clinical pipeline.

In some embodiments, a gene of interest of the disclosure is related to PMF. In some embodiments, a gene of interest is related to biomolecule production. In some embodiments, a gene of interest encodes a biomolecule that is to be harvested from the plant tissue, e.g., for pharmaceutical and/or industrial purposes. In some embodiments, the biomolecule is a lipid, a nucleic acid, a protein, or a carbohydrate. In some embodiments, the biomolecule is a biopharmaceutical, e.g., a vaccine antigen or a monoclonal antibody. In some embodiments, the gene of interest encodes a non-pharmaceutical biomolecule.

Tables 2-4 contain illustrative biomolecules that are encoded by genes of interest of the disclosure in some embodiments. In some embodiments, these biomolecules are for production within a plant of the disclosure via a genetic construct of the disclosure.

TABLE 2
Illustrative vaccine candidates for expression in plants.
Vaccine Candidates

| Recombinant | | | | |
|---|---|---|---|---|
| Protein (Gene | | Expression | Transformation | Expression |
| of interest) | Pathogen/Disease | System | Method | Level |
| Hepatitis B | Hepatitis B virus | Tobacco | Agrobacterium | 66 ng/mg of |
| surface antigen | | (Nicotiana | mediated (Stable | soluble protein |
| | | tabacum) | expression/Nucleus) | |
| Structural | Rabbit | Potato | Agrobacterium | 0.3% of total |
| protein VP60 | hemorrhagic | (Solanum | mediated (Stable | soluble protein |
| | disease virus | tuberosum) | expression/Nucleus) | |
| | (RHDV) | | | |
| Spike (S) protein | Transmissible | Tobacco | Agrobacterium | 0.1-0.2% of |
| of transmissible | gastroenteritis | (Nicotiana | mediated (Stable | total soluble |
| gastroenteritis | virus (TGEV) | tabacum) | expression/Nucleus) | protein |
| virus | | | | |
| Vaccine Candidates | | | | |
| Hemagglutinin | Rinderpest virus | Peanut (Arachis | Agrobacterium | 0.2-1.3% of |
| protein of | (RPV) | hypogea L.) | mediated (Stable | total soluble |
| rinderpest virus | | | expression/Nucleus) | protein |
| Glycoprotein D | Bovine herpes | Tobacco | Mechanical inoculation | 20 μg/g fresh |
| (gD) of bovine | virus | (Nicotiana | (Stable expression/ | weight (FW) |
| herpes virus | | benthamiana) | Nucleus) | |
| LI major capsid | Human | Tobacco | Agrobacterium | 2-4 μg/kg FW |
| protein | papillomavirus | (Nicotiana | mediated (Stable | |
| | | tabacum) | expression/Nucleus) | |
| Spike (S) protein | Transmissible | Corn (Zea | Agrobacterium | 13 mg/kg FW |
| of transmissible | gastroenteritis | mays) | mediated (Stable | |
| gastroenteritis | virus (TGEV) | | expression/Nucleus) | |
| virus | | | | |
| Spike (S) protein | Infectious | Potato | Agrobacterium | 2.39-2.53 μg/g |
| of infectious | bronchitis virus | (Solanum | mediated (Stable | FW |
| bronchitis virus | (IBV) | tuberosum) | expression/Nucleus) | |

TABLE 3
Illustrative antibodies and fragments thereof for expression in plants.
Antibodies

| Recombinant | | | | |
|---|---|---|---|---|
| Protein (Gene | | Expression | Transformation | Expression |
| of interest) | Pathogen/Disease | System | Method | Level |
| cT84.66 | Cancer (tumor | Tobacco | Agrobacterium | 1 mg/kg FW |
| | marker) | (Nicotiana | mediated (Transient | |
| | | tabacum) | expression) | |
| scFVT84.66 | Cancer (tumor | Tobacco | Agrobacterium | 5 mg/kg FW |
| | | (Nicotiana | mediated (Transient | |
| | marker) | tabacum) | expression) | |
| scFVT84.66 | Cancer (tumor | Rice (Oryza | Biolistic method | 3.8 μg/g FW |
| | marker) | sativa) | (Stable expression/ | |
| | | | Nucleus) | |
| scFVT84.66 | Cancer (tumor | Cereal crops | Biolistic method | 30 μg/g FW |
| | marker) | (wheat and rice) | (Stable expression/ | |
| | | | Nucleus) | |
| BR55-2 | Human colorectal | Tobacco | Agrobacterium | 30 mg/kg FW |
| | cancer | (Nicotiana | mediated (Stable | |
| | | tabacum) | expression/Nucleus) | |
| 2F5 | HIV | Tobacco | Agrobacterium | 0.01% of total |
| | | (Nicotiana | mediated (Stable | soluble protein |
| | | benthamiana) | expression/Nucleus) | |
| 2G12 | HIV | Tobacco | Agrobacterium | 0.3 g/kg FW |
| | | (Nicotiana | mediated (Transient | |
| | | benthamiana) | expression) | |
| 2G12 | HIV | Tobacco | Agrobacterium | 8 mg/L culture |
| | | (Nicotiana | mediated (Stable | medium |
| | | tabacum) | expression/Nucleus) | |

TABLE 4
Additional illustrative biomolecules for plant expression.

| Recombinant Protein | | Transformation | Expression |
|---|---|---|---|
| (Gene of interest) | Expression System | Method | Level |
| Human serum albumin | Potato (Solanum | Agrobacterium | 0.25 μg/mg (0.02% of |
| | tuberosum) | mediated (Stable | total soluble protein) |
| | | expression/Nucleus) | |
| Erythropoietin | Tobacco (Nicotiana | Agrobacterium | 26 pg/mg total |
| | tabacum) | mediated (Stable | protein |
| | | expression/Nucleus) | |
| α1-antitrypsin | Rice (Japonica rice) | Biolistic method | 4.6-5.7 mg/g dry cell |
| | | (Stable | |
| | | expression/Nucleus) | |
| Aprotinin | Corn | Biolistic method | 0.069% of total |
| | | (Stable | extractable seed |
| | | expression/Nucleus) | protein |
| Human-secreted | Tobacco (Nicotiana | Agrobacterium | 1.1 μg/g FW (3% of |
| alkaline | tabacum) | mediated (Stable | total soluble protein) |
| phosphatase | | expression/Nucleus) | |
| Collagen | Tobacco (Nicotiana | Agrobacterium | 0.03 g/kg powdered |
| | tabacum) | mediated (Stable | plants |
| | | expression/Nucleus) | |
| Human | Tobacco | Biolistic method | >7% of total soluble |
| somatotropin | | (Stable | protein |
| (hST) | | expression/Chloroplast) | |
| Bacillus | Tobacco | Biolistic method | 5 mg/g FW (45.3- |
| thuringiensis | | (Stable | 46.1% of total soluble |
| (Bt) cry2Aa2 | | expression/Chloroplast) | protein) |
| Human serum | Tobacco (Nicotiana | Biolistic method | 11.1% of total protein |
| albumin | tabacum) | (Stable | |
| | | expression/Chloroplast) | |

### Additional Traits

Additional traits can be introduced via a genetic construct of the present invention. A non-limiting example of such a trait is a coding sequence which decreases RNA and/or protein levels. The decreased RNA and/or protein levels may be achieved through RNAi methods, such as those described in U.S. Pat. No. 6,506,559.

Another trait that may be incorporated into a genetic construct of the disclosure is a sequence which allows for site-specific recombination. Examples of such sequences include the FRT sequence used with the FLP recombinase (Zhu and Sadowski, J. Biol. Chem., 270:23044-23054, 1995) and the LOX sequence used with CRE recombinase (Sauer, Mol. Cell. Biol., 7:2087-2096, 1987). The recombinase genes can be encoded at any location within the genome of a plant of the disclosure and are active in a hemizygous state.

In certain embodiments, plants may be made more tolerant to or more easily transformed with Agrobacterium tumefaciens. For example, expression of p53 and iap, two baculovirus cell-death suppressor genes, inhibited tissue necrosis and DNA cleavage. Additional targets may include plant-encoded proteins that interact with the Agrobacterium Vir genes: enzymes involved in plant cell wall formation; and histones, histone acetyltransferases and histone deacetylases (reviewed in Gelvin, Microbiology & Mol. Biol. Reviews. 67:16-37, 2003).

In addition to the modification of oil, fatty acid, or phytate content described above, certain embodiments may modify the amounts or levels of other compounds. For example, the amount or composition of antioxidants can be altered. See, for example, U.S. Pat. Nos. 6,787,618 and 7,154,029 and International Patent Application Publication No. WO 00/68393. which disclose the manipulation of antioxidant levels, and International Patent Application Publication No. WO 03/082899. which discloses the manipulation of an antioxidant biosynthetic pathway.

Additionally, seed amino acid content may be manipulated. U.S. Pat. No. 5,850.016 and International Patent Application Publication No. WO 99/40209 disclose the alteration of the amino acid compositions of seeds. U.S. Pat. Nos. 6,080,913 and 6,127,600 disclose methods of increasing accumulation of essential amino acids in seeds.

U.S. Pat. No. 5,559,223 describes synthetic storage proteins of which the levels of essential amino acids can be manipulated. International Patent Application Publication No. WO 99/29882 discloses methods for altering amino acid content of proteins. International Patent Application Publication No. WO 98/20133 describes proteins with enhanced levels of essential amino acids. International Patent Application Publication No. WO 98/56935 and U.S. Pat. Nos. 6,346,403; 6,441,274; and 6,664,445 disclose plant amino acid biosynthetic enzymes. International Patent Application Publication No. WO 98/45458 describes synthetic seed proteins having a higher percentage of essential amino acids than wild-type.

U.S. Pat. No. 5,633,436 discloses plants comprising a higher content of sulfur-containing amino acids; U.S. Pat. No. 5,885,801 discloses plants comprising a high threonine content; U.S. Pat. Nos. 5,885,802 and 5,912,414 disclose plants comprising a high methionine content; U.S. Pat. No. 5,990,389 discloses plants comprising a high lysine content, U.S. Pat. No. 6,459,019 discloses plants comprising an increased lysine and threonine content, International Patent Application Publication No. WO 98/42831 discloses plants comprising a high lysine content, International Patent Application Publication No. WO 96/01905 discloses plants comprising a high threonine content, and International Patent Application Publication No. WO 95/15392 discloses plants comprising a high lysine content.

### Host Factor

In some embodiments, a host factor is a plant gene. In some embodiments, the host factor sequence is identical to a host factor gene, is a modified version of a host factor gene, is a part of a host factor gene, or is homologous to some part of a host factor gene. In some embodiments, the host factor sequence is a non-coding sequence with significant homology to a host factor gene, such that silencing of the host factor sequence will likely lead to silencing of the host factor gene within the plant cell.

In some embodiments, the host factor is necessary for plant fertility. For example, in some embodiments, silencing of the host factor leads to sterility. In some embodiments, the host factor is necessary for survival. For example, in some embodiments, silencing of the host factor leads to death. In some embodiments, the host factor is necessary for maintenance of homeostasis. In some embodiments, the host factor is involved in silencing of invasive genomic elements. For example, in some embodiments, the host factor is involved in silencing, such that silencing of the host factor leads to suppression of silencing. In some embodiments, the host factor is involved in chemical resistance, such that its loss re-introduces susceptibility to the chemical.

With the use of the constructs herein, in the event that the host targets the construct for silencing. the host factor will also be targeted for silencing and prevent the host from passing on the phenotype. In the case where the host factor is a gene involved in the silencing of invasive genomic elements, in the instances where silencing occurs towards the transgenic construct, the machinery involved in the silencing is also targeted, thus reducing the silencing of the genetic construct, the genetic cargo, or the gene of interest, and increasing transposable element activity globally threatening genomic stability.

### Plant Gene Silencing-Related Host Factor Sequences

In some embodiments, the host factor is involved in a host plant gene silencing pathway. In some embodiments, the host factor is involved with recognition of sequences for silencing, repression of translation, degradation of target RNAs, site-specific DNA methylation, or post-transcriptional RNA regulation.

In some embodiments, the host factor is an Argonaute (AGO) family protein. In some embodiments, the host factor is an AGO1 protein. In some embodiments, the host factor is an AGO4 protein. In some embodiments, the host factor is a dicer protein or a dicer-like (DCL) protein. In some embodiments, the host factor is DCL1. In some embodiments, the host factor is an RNA methyltransferase. In some embodiments, the host factor is HUA Enhancer1 (HEN1). In some embodiments, the host factor is involved in RNA-dependent DNA methylation. In some embodiments, the host factor is an RNA-dependent RNA polymerase (RdRp). In some embodiments, the host factor is a part of the Pol V complex. In some embodiments, the host factor is involved in transcriptional silencing of repetitive loci by RdDM. In some embodiments, the host factor is a nuclear RNA helicase with involved in sRNA biogenesis.

### siRNA

In some embodiments, the host factor sequence comprises an siRNA sequence to bias silencing toward the host factor sequence.

In some embodiments, the siRNA sequence is incorporated into an intron in the host factor sequence. In some embodiment, the siRNA sequence is incorporated into an exon in the host factor sequence. In some embodiments, the siRNA sequence is incorporated into a promoter.

In some embodiments, the genetic construct comprises multiple siRNA sequences. In some embodiments, the host factor sequence comprises multiple siRNA sequences. In some embodiments, the multiple siRNA sequences are in the same stretch of nucleic acids. In some embodiments, the multiple siRNA sequences are in separate stretches of nucleic acids. In some embodiments, the multiple siRNA sequences are the same sequence. In some embodiments, the multiple siRNA sequences are different sequences. In some embodiments, the multiple siRNA sequences are incorporated into various positions along the host factor sequence to allow for multiple targets for silencing.

In some embodiments, the siRNA sequences are endogenous plant siRNA sequences. In some embodiments, the siRNA sequences are identified for inclusion from known genome-wide profiling of siRNA sequences endogenous to the host plant. In some embodiments, the siRNA sequences are included by way of inclusion of a known silenced region, e.g., a silenced transposable element sequence that comprises multiple siRNA sequences. For example, in some embodiments, the genetic construct comprises a stretch of 30, 40, 50, 60, 70, 80, 90, 100, 150, 200, or 250 nucleotides of a silenced transposable element sequence believed to comprise multiple siRNA sequences.

### Vectors

The present disclosure provides a vector comprising a genetic construct of the disclosure. In some embodiments, the vector is a viral vector. In some embodiments, the vector is a bacterial vector. In some embodiments, the vector is a polynucleotide. In some embodiments, the vector is a plasmid. In some embodiments, the vector is a CRISPR-based vector. In some embodiments, the vector is a retrotransposon-based vector. In some embodiments, the vector comprises the means for incorporation of the genetic constructs disclosed herein. In some embodiments, the vector is a vector as described herein for plant transformation or plant breeding purposes.

### Expression Vectors for Transformation: Marker Genes

Expression vectors usually include at least one genetic marker, operably linked to a regulatory element (a promoter, for example) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or an herbicide, or genes that encode an altered target which is insensitive to the inhibitor. A few positive selection methods are also known in the art.

One commonly used selectable marker gene for plant transformation is the neomycin phosphotransferase II (nptII) gene which, when under the control of plant regulatory signals, confers resistance to kanamycin. Fraley et al., Proc. Natl. Acad. Sci. U.S.A., 80:4803 (1983). The aminoglycoside phosphotransferases APH(3′)II and APH(3′)I carried by transposons TnS and Tn601 respectively were shown to inactivate the related aminoglycoside antibiotics G418), neomycin and kanamycin (Davies and Smith, 1978; Jimenez and Davies, 1980). The kanamycin resistance (KmR) gene from Staphylococcus aureus, which are used for the present disclosure, was sequenced and identified when compared with similar genes isolated from Streptomyces fradiae and from two transposons, Tn5 and Tn903, originally isolated from Klebsiella pneumoniae and Salmonella typhimurium, respectively (Gray and Fitch, 1983). Another commonly used selectable marker gene is the hygromycin phosphotransferase gene which confers resistance to the antibiotic hygromycin. Vanden Elzen et al., Plant Mol. Biol., 5:299 (1985).

Additional selectable marker genes of bacterial origin that confer resistance to antibiotics include gentamycin acetyl transferase, streptomycin phosphotransferase and aminoglycoside-3′-adenyl transferase. the bleomycin resistance determinant. Hayford et al., Plant Physiol. 86:1216 (1988), Jones et al., Mol. Gen. Genet., 210:86 (1987). Svab et al., Plant Mol. Biol. 14:197 (1990) Hille et al., Plant Mol. Biol. 7:171 (1986). Other selectable marker genes confer resistance to herbicides such as glyphosate, glufosinate or bromoxynil. Comai et al., Nature 317:741-744 (1985). Gordon-Kamm et al., Plant Cell 2:603-618 (1990) and Stalker et al., Science 242:419-423 (1988).

Selectable marker genes for plant transformation not of bacterial origin include, for example, mouse dihydrofolate reductase, plant 5-enolpyruvylshikimate-3-phosphate synthase and plant acetolactate synthase. Eichholtz et al., Somatic Cell Mol. Genet. 13:67 (1987). Shah et al., Science 233:478 (1986), Charest et al., Plant Cell Rep. 8:643 (1990).

Another class of marker genes for plant transformation requires screening of presumptively transformed plant cells rather than direct genetic selection of transformed cells for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Commonly used genes for screening presumptively transformed cells include beta-glucuronidase (GUS), beta-galactosidase, luciferase and chloramphenicol acetyltransferase. Jefferson, R. A., Plant Mol. Biol. Rep. 5:387 (1987). Tecri et al., EMBO J. 8:343 (1989). Koncz et al., Proc. Natl. Acad. Sci. USA 84:131 (1987), DeBlock et al., EMBO J. 3:1681 (1984). In vivo methods for visualizing GUS activity that do not require destruction of plant tissue are available. Molecular Probes publication 2908, IMAGENE GREEN, p. 1-4 (1993) and Naleway et al., J. Cell Biol. 115:151a (1991). However, these in vivo methods for visualizing GUS activity have not proven useful for recovery of transformed cells because of low sensitivity, high fluorescent backgrounds and limitations associated with the use of luciferase genes as selectable markers.

In some aspects, a gene encoding Green Fluorescent Protein (GFP) has been utilized as a marker for gene expression in prokaryotic and eukaryotic cells. Chalfic et al., Science 263:802 (1994). GFP and mutants of GFP may be used as screenable markers.

### Expression Vectors for Transformation: Promoters Genes included in expression vectors are typically driven by a nucleotide sequence comprising a

regulatory element, for example, a promoter. Several types of promoters are well known in the transformation arts as are other regulatory elements that can be used alone or in combination with promoters.

As used herein, “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred”. Promoters that initiate transcription only in a certain tissue are referred to as “tissue-specific”. A “cell-type” specific promoter primarily drives expression in certain cell types in one or more organs, for example, vascular cells in roots or leaves. An “inducible” promoter is a promoter which is under environmental control. Examples of environmental conditions that may affect transcription by inducible promoters include anaerobic conditions or the presence of light. Tissue-specific, tissue-preferred, cell type specific, and inducible promoters constitute the class of “non-constitutive” promoters. A “constitutive” promoter is a promoter that is active under most environmental conditions, and cell types.

### A. Inducible Promoters

An inducible promoter is operably linked to a gene for expression in a plant. Optionally, the inducible promoter is operably linked to a nucleotide sequence encoding a signal sequence which is operably linked to a gene for expression in a plant. With an inducible promoter the rate of transcription increases in response to an inducing agent.

Any inducible promoter can be used in the instant disclosure. See Ward et al., Plant Mol. Biol. 22:361-366 (1993). Exemplary inducible promoters include, but are not limited to, that from the ACEI system which responds to copper (Mett et al., PNAS 90:4567-4571 (1993)): In2 gene from maize which responds to benzenesulfonamide herbicide safeners (Hershey et al., Mol. Gen Genetics 227:229-237 (1991) and Gatz et al., Mol. Gen. Genetics 243:32-38 (1994)) or Tet repressor from Tn10 (Gatz et al., Mol. Gen. Genetics 227:229-237 (1991)). A particularly preferred inducible promoter is a promoter that responds to an inducing agent to which plants do not normally respond. An exemplary inducible promoter is the inducible promoter from a steroid hormone gene, the transcriptional activity of which is induced by a glucocorticosteroid hormone. Schena et al., Proc. Natl. Acad. Sci. USA 88:0421 (1991).

### B. Constitutive Promoters

A constitutive promoter is operably linked to a gene for expression in a plant or the constitutive promoter is operably linked to a nucleotide sequence encoding a signal sequence which is operably linked to a gene for expression in a plant.

Many different constitutive promoters can be utilized in the instant disclosure. Exemplary constitutive promoters include, but are not limited to, the promoters from plant viruses such as the 35S promoter from CaMV (Odell et al., Nature 313:810-812 (1985)) and the promoters from such genes as rice actin (McElroy et al., Plant Cell 2:163-171 (1990)): ubiquitin (Christensen et al., Plant Mol. Biol. 12:619-632 (1989) and Christensen et al., Plant Mol. Biol. 18:675-689 (1992)): pEMU (Last et al., Theor. Appl. Genet. 81:581-588 (1991)): MAS (Velten et al., EMBO J. 3:2723-2730 (1984)) and maize H3 histone (Lepetit et al., Mol. Gen. Genetics 231:276-285 (1992) and Atanassova et al., Plant Journal 2 (3): 291-300 (1992)).

The ALS promoter, Xbal/Ncol fragment 5′ to the Brassica napus ALS3 structural gene (or a nucleotide sequence similarity to said Xbal/Ncol fragment), represents a particularly useful constitutive promoter. See PCT application WO 96/30530.

### C. Tissue-Specific or Tissue-Preferred Promoters

A tissue-specific promoter is operably linked to a gene for expression in a plant. Optionally, the tissue-specific promoter is operably linked to a nucleotide sequence encoding a signal sequence which is operably linked to a gene for expression in a plant. Plants transformed with a gene of interest operably linked to a tissue-specific promoter produce the protein product of the transgene exclusively, or preferentially, in a specific tissue.

Any tissue-specific or tissue-preferred promoter can be utilized in the instant disclosure. Exemplary tissue-specific or tissue-preferred promoters include, but are not limited to, a root-preferred promoter—such as that from the phascolin gene (Murai et al., Science 23:476-482 (1983) and Sengupta-Gopalan et al., Proc. Natl. Acad. Sci. USA 82:3320-3324 (1985)); a leaf-specific and light-induced promoter such as that from cab or rubisco (Simpson et al., EMBO J. 4 (11):2723-2729 (1985) and Timko et al., Nature 318:579-582 (1985)); an anther-specific promoter such as that from LAT52 (Twell et al., Mol. Gen. Genetics 217:240-245 (1989)); a pollen-specific promoter such as that from Zm13 (Guerrero et al., Mol. Gen. Genetics 244:161-168 (1993)) or a microspore-preferred promoter such as that from apg (Twell et al., Sex. Plant Reprod. 6:217-224 (1993)).

### Expression Vectors for Transformation: Terminators

As used herein, the term “terminator” or “termination sequence” generally refers to a 3′ flanking region of a gene that contains nucleotide sequences which regulate transcription termination and typically confer RNA stability.

The disclosure provides terminator sequences that find use in proper transcriptional processing of recombinant nucleic acids in the transformation vectors taught herein. Although terminator sequences do not by themselves initiate gene transcription, their presence can increase accurate processing and termination of the RNA transcript, and result in message stability. The use of recombinant terminator sequences is established in the art. It is appreciated that an understanding of the molecular mechanisms underlying terminator sequence activity are not required to make or use the present disclosure.

### Plant Transformation

In some embodiments, plants of the present disclosure are modified by introducing one or more transgenes which when expressed lead to desired phenotypes. In some embodiments, new genetic material is introduced into a plant genome via the use of living cells of the bacterial pathogen Agrobacterium tumefaciens to inject a piece of DNA, called transfer or T-DNA, into individual plant cells (usually following wounding of the tissue) where it is targeted to the plant nucleus for chromosomal integration.

Numerous methods for plant transformation have been developed and are well known in the art, including biological and physical plant transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press. Inc. Boca Raton, 1993) pages 67-88. In addition, expression vectors and in-vitro culture methods for plant cell or tissue transformation and regeneration of plants are available. See, for example, Gruber et al., “Vectors for Plant Transformation” in Methods in Plant Molecular Biology and Biotechnology, Glick. B. R. and Thompson, J. E. Eds. (CRC Press. Inc., Boca Raton, 1993) pages 89-119.

### A. Agrobacterium-Mediated Transformation

One method for introducing an expression vector into plants is based on the natural transformation system of Agrobacterium. See, for example, Horsch et al., Science 227:1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado. C. I., Crit. Rev. Plant Sci. 10:1 (1991). Descriptions of Agrobacterium vector systems and methods for Agrobacterium-mediated gene transfer are provided by Gruber et al., supra. Miki et al., supra and Moloney et al., Plant Cell Reports 8:238 (1989). See also. U.S. Pat. No. 5,563,055 (Townsend and Thomas), issued Oct. 8, 1996. There are numerous patents governing Agrobacterium mediated transformation and particular DNA delivery plasmids designed specifically for use with Agrobacterium—for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. No. 5,731,179, EP068730, WO9516031, U.S. Pat. Nos. 5,693,512, 6,051,757 and EP904362A1, which are all hereby incorporated by reference in their entirety.

Agrobacterium-mediated plant transformation involves as a first step the placement of DNA fragments cloned on plasmids into living Agrobacterium cells, which are then subsequently used for transformation into individual plant cells. Agrobacterium-mediated plant transformation is thus an indirect plant transformation method. Alternatively, Agrobacterium-mediated plant transformation involves cloning DNA fragments into the disarmed Ti or Vi plasmid of Agrobacterium, such as described in Collier (2018), and using the resulting engineered Agrobacterium for plant transformation.

Agrobacterium-mediated transformation is achieved through the use of a genetically engineered soil bacterium belonging to the genus Agrobacterium. Several Agrobacterium species mediate the transfer of a specific DNA known as “T-DNA” that can be genetically engineered to carry any desired piece of DNA into many plant species. The major events marking the process of T-DNA mediated pathogenesis are: induction of virulence genes, processing and transfer of T-DNA. This process is the subject of many reviews (Ream. 1989; Howard and Citovsky, 1990; Kado, 1991; Hooykaas and Schilperoort, 1992; Winnans, 1992; Zambryski, 1992; Gelvin, 1993; Binns and Howitz, 1994; Hooykaas and Beijersbergen 1994; Lessl and Lanka, 1994; Zupan and Zambryski, 1995).

Agrobacterium-mediated genetic transformation of plants involves several steps. The first step, in which the Agrobacterium and plant cells are first brought into contact with each other, is generally called “inoculation”. Following the inoculation step, the Agrobacterium and plant cells/tissues are usually grown together for a period of several hours to several days or more under conditions suitable for growth and T-DNA transfer. This step is termed “co-culture”. Following co-culture and T-DNA delivery, the plant cells are often treated with bacteriocidal and-or bacteriostatic agents to kill the Agrobacterium.

Although transgenic plants produced through Agrobacterium-mediated transformation generally contain a simple integration pattern as compared to microparticle-mediated genetic transformation, a wide variation in copy number and insertion patterns exists (Jones et al. 1987: Jorgensen et al., 1987). Moreover, even within a single plant genotype, different patterns of transfer DNA integration are possible based on the type of explant and transformation system used (Grevelding et al., 1993). Factors that regulate transfer DNA copy number are poorly understood.

### B. Direct Gene Transfer

Direct plant transformation methods using DNA have also been reported. The first of these to be reported historically is electroporation, which utilizes an electrical current applied to a solution containing plant cells (M. E. Fromm et al., Nature, 319, 791 (1986); H. Jones et al., Plant Mol. Biol., 13, 501 (1989) and H. Yang et al., Plant Cell Reports, 7, 421 (1988).

Another direct method, called “biolistic bombardment”, uses ultrafine particles, usually tungsten or gold, that are coated with DNA and then sprayed onto the surface of a plant tissue with sufficient force to cause the particles to penetrate plant cells, including the thick cell wall, membrane and nuclear envelope, but without killing at least some of them (U.S. Pat. Nos. 5,204,253, 5,015,580).

A third direct method uses fibrous forms of metal or ceramic consisting of sharp, porous or hollow needle-like projections that literally impale the cells, and also the nuclear envelope of cells. Both silicon carbide and aluminum borate whiskers have been used for plant transformation (Mizuno et al., 2004; Petolino et al., 2000: U.S. Pat. No. 5,302,523 U.S. Application 20040197909) and also for bacterial and animal transformation (Kaepler et al., 1992; Raloff, 1990; Wang., 1995). There are other methods reported, and undoubtedly, additional methods will be developed. The methods taught herein are capable of detecting the non-naturally occurring nucleotide junctions that result from any plant transformation method.

The sequences of the present disclosure may be transferred to any cell, for example, such as a plant cell transformation competent bacterium. Such bacteria are known in the art and may, for instance, belong to the following species: Agrobacterium spp., Rhizobium spp., Sinorhizobium spp., Mesorhizobium spp., Phyllobacterium spp. Ochrobactrum spp. and Bradyrhizobium spp. In some embodiments, such bacteria may belong to Agrobacterium spp.

The present disclosure also relates to a plant cell transforming bacterium comprising the sequences disclosed herein, and which may be used for transforming a plant cell. In some embodiments, the plant transforming bacteria is selected from Agrobacterium spp., Rhizobium spp., Sinorhizobium spp., Mesorhizobium spp., Phyllobacterium spp. Ochrobactrum spp. or Bradyrhizobium spp.

The present disclosure also relates to a method for transforming a plant cell comprising: contacting at least a first plant cell with a plant cell transforming bacteria of the present disclosure; and selecting at least a plant cell transformed with one or more of the sequences disclosed herein. In one embodiment, a method of the disclosure further comprises regenerating a plant from the plant cell.

In some embodiments, the present disclosure provides a method for transforming a plant cell, wherein the method comprises: (i) introducing a plant transformation vector taught herein into the plant cell; and (ii) cultivating the transformed plant cell under conditions conducive to regeneration and mature plant growth.

The foregoing methods for transformation would typically be used for producing a transgenic variety. The transgenic variety could then be crossed with another (non-transformed or transformed) variety in order to produce a new transgenic variety. Alternatively, a genetic trait that has been engineered into a particular plant line using the foregoing transformation techniques could be moved into another line using traditional backcrossing techniques that are well known in the plant breeding arts. For example, a backcrossing approach could be used to move an engineered trait from a public, non-elite variety into an elite variety, or from a variety containing a foreign gene in its genome into a variety or varieties that do not contain that gene. As used herein, “crossing” can refer to a simple X by Y cross or the process of backcrossing depending on the context.

Persons of ordinary skill in the art will recognize that plants comprising the sequences disclosed herein also includes derivative varieties that retain the essential distinguishing characteristics of the event in question, such as a locus converted plant of that variety or a transgenic derivative having one or more value-added genes incorporated therein (such as herbicide or pest resistance).

Likewise, transgenes can be introduced into the plant using any of a variety of established recombinant methods well-known to persons skilled in the art, such as: Gressel, 1985, Biotechnologically Conferring Herbicide Resistance in Crops: The Present Realities. In Molecular Form and Function of the Plant Genome. L. van Vloten-Doting, (ed.), Plenum Press, New York; Huttner, S. L., et al., 1992, Revising Oversight of Genetically Modified Plants, Bio/Technology; Klee, H., et al., 1989, Plant Gene Vectors and Genetic Transformation: Plant Transformation Systems Based on the use of Agrobacterium tumefaciens, Cell Culture and Somatic Cell Genetics of Plants; Koncz, C., et al., 1986. The Promoter of T. sub.L-DNA Gene 5 Controls the Tissue-Specific Expression of Chimeric Genes Carried by a Novel Type of Agrobacterium Binary Vector; Molecular and General Genetics; Lawson. C., et al., 1990, Engineering Resistance to Mixed Virus Infection in a Commercial Potato Cultivar; Resistance to Potato Virus X and Potato Virus Y in Transgenic Russet Burbank, Bio/Technology; Mitsky, T. A., et al., 1996, Plants Resistant to Infection by PLRV. U.S. Pat. No. 5,510,253; Newell, C. A., et al., 1991, Agrobacterium-Mediated Transformation of Solanum tuberosum L. Cv. Russet Burbank. Plant Cell Reports; Perlak. F. J., et al., 1993, Genetically Improved Potatoes: Protection from Damage by Colorado Potato Beetles, Plant Molecular Biology; all of which are incorporated herein by reference for this purpose.

Methods of modifying nucleic acid constructs to increase expression levels in plants are also generally known in the art (see, e.g. Rogers et al., 260 J. Biol. Chem. 3731-38, 1985; Cornejo et al., 23 Plant Mol. Biol. 567:81,1993). In engineering a plant system to affect the rate of transcription of a protein, various factors known in the art, including regulatory sequences such as positively or negatively acting sequences, enhancers and silencers, as well as chromatin structure may have an impact. The present disclosure provides that at least one of these factors may be utilized in engineering plants to express a protein of interest. The sequences of the present disclosure are native genetic elements, i.e., are isolated from the selected plant species to be modified.

### Transgene Stacking Using the GAANTRY System

The GAANTRY system (Gene Assembly in Agrobacterium by Nucleic acid Transfer using Recombinase technologY) leverages recombinase-mediated stacking technology. The specificity and efficiency of recombinases make them extremely attractive for genome engineering. Advancements in molecular biology and recombinases have paved the way for gene stacking with the assistance of unidirectional recombination systems. Development of this high-efficiency gene stacking system uses the specificity of the recombinases to effectively deliver the target genes of interest to a predetermined position. This is a flexible and effective system for stably stacking multiple genes within an Agrobacterium virulence plasmid Transfer-DNA (T-DNA).

Agrobacterium-mediated transformation of plants with one or a few genes is relatively routine, but the assembly and transformation of large constructs carrying multiple genes and their efficient use to generate high-quality transgenic plants has been a challenge.

The present disclosure teaches GAANTRY, which is a flexible and effective system for stably stacking multiple genes within an Agrobacterium virulence plasmid Transfer-DNA (T-DNA). This GAANTRY system is well described in Collier, R. et al (2018), Plant Journal 95. 573-583 and McCue et al (2019) BMC Research Notes 12, 457, each of which is incorporated herein by reference for all purposes.

The GAANTRY system is based on the combined use of unidirectional integration and excision controlled by three site-specific serine recombinases, which is an effective and stable system for stacking multiple genes within an Agrobacterium virulence plasmid T-DNA. The gene stacking system utilizes ‘P-Donor’ and ‘B-Donor’ cloning vectors, and ‘P-Helper’ and ‘B-Helper’ vectors, for the insertion of sequences of interest. The P-Donor and B-Donor vectors contain either attP or attB, respectively, recognition sites enabling precise integration into the virulence plasmid of the GAANTRY ArPORTI strain. Plant transformation with T-DNA expressed from a GAANTRY modified Agrobacterium can produce high quality events that contain low copies of a complete T-DNA with limited incorporation of vector ‘backbone’ sequences (Collier, 2018).

The resulting engineered Agrobacterium strain can be directly used for plant transformation. The gene stacking strategy is efficient, precise, modular, and allows control over the orientation and order in which genes are stacked within the T-DNA.

### Plants for use With the Disclosed Methods

Also disclosed herein are plants or plant parts comprising the constructs or vectors of the disclosure.

In some embodiments, the plant parts are plant cells, plant protoplasts, or plant cell tissue cultures from which plants can be regenerated, plant calli, plant clumps and plant cells that are intact in plants or parts of plants, such as embryos, pollens, ovules, flowers, seeds, heads, rootstocks, scions, stems, roots, anthers, pistils, root tips, leaves, meristematic cells, axillary buds, hypocotyls, cotyledons, ovaries, seed coats, endosperms and the like. In some embodiments, the plant part comprises at least one cell of said plant. In some embodiments, the plant part is further defined as a pollen, a meristem, a cell or an ovule. In some embodiments, a plant regenerated from the plant part has all of the phenotypic and morphological characteristics of a plant of the present disclosure.

In some embodiments, plant tissues are from plants that are important or interesting for agriculture, horticulture, biomass for the production of biofuel molecules and other chemicals, and/or forestry. Some examples of these plants may include pineapple, banana, coconut, lily, grasspeas and grass; and dicotyledonous plants, such as, for example, peas, alfalfa, tomatillo, melon, chickpea, chicory, clover, kale, lentil, soybean, tobacco, potato, sweet potato, radish, cabbage, rape, apple trees, grape, cotton, sunflower, thale cress, canola, citrus (including orange, mandarin, kumquat, lemon, lime, grapefruit, tangerine, tangelo, citron, and pomelo), Nicotiana tabacum (tobacco), Lupinus albus (lupin), Uniola paniculata (oats), Hordeum vulgare (barley), and Lolium spp. (rye).

In some embodiments, plant tissues or plant parts, e.g., seeds, from a monocotyledonous plant are treated. Monocotyledonous plants belong to the orders of the Alismatales, Arales, Arecales, Bromeliales, Commelinales, Cyclanthales, Cyperales, Eriocaulales, Hydrocharitales, Juncales, Lilliales, Najadales, Orchidales, Pandanales, Poales, Restionales, Triuridales, Typhales, and Zingiberales. Plants belonging to the class of the Gymnospermae are Cycadales, Ginkgoales, Gnetales, and Pinales. In some embodiments, the monocotyledonous plant can be selected from the group consisting of a maize, rice, wheat, barley, and sugarcane.

In some embodiments, plant tissues or plant parts, e.g., seeds, from a dicotyledonous plant are treated, including those belonging to the orders of the Aristochiales, Asterales, Batales, Campanulales, Capparales, Caryophyllales, Casuarinales, Celastrales, Cornales, Diapensales, Dilleniales, Dipsacales, Ebenales, Ericales, Eucomiales, Euphorbiales, Fabales, Fagales, Gentianales, Geraniales, Haloragales, Hamamelidales, Middles, Juglandales, Lamiales, Laurales, Lecythidales, Leitneriales, Magniolales, Malvales, Myricales, Myrtales, Nymphacales, Papeverales, Piperales, Plantaginales, Plumb aginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Theales, Trochodendrales, Umbellales, Urticales, and Violates. In some embodiments, the dicotyledonous plant can be selected from the group consisting of cotton, soybean, pepper, and tomato.

### Plant Breeding

The goal of plant breeding is to develop new, unique and superior plant cultivars and hybrids. The breeder initially selects and crosses two or more parental lines, followed by repeated selfing and selection, producing many new genetic combinations. Another method used to develop new, unique and superior cultivars occurs when the breeder selects and crosses two or more parental lines followed by haploid induction and chromosome doubling that result in the development of dihaploid cultivars. The breeder can theoretically generate billions of different genetic combinations via crossing, selfing and mutations and the same is true for the utilization of the dihaploid breeding method.

Each year, the plant breeder selects the germplasm to advance to the next generation. This germplasm is grown under unique and different geographical, climatic and soil conditions, and further selections are then made, during and at the end of the growing season. The cultivars developed are unpredictable. This unpredictability is because the breeder's selection occurs in unique environments, with no control at the DNA level (using conventional breeding procedures or dihaploid breeding procedures), and with millions of different possible genetic combinations being generated. A breeder of ordinary skill in the art cannot predict the final resulting cultivars the breeder develops, except possibly in a very gross and general fashion. This unpredictability results in the expenditure of large research monies to develop superior new cultivars.

The development of commercial cultivar requires the development and selection of plants, the crossing of these plants, and the evaluation of the crosses.

Pedigree breeding and recurrent selection breeding methods are used to develop cultivars from breeding populations. Breeding programs combine desirable traits from two or more cultivars or various broad-based sources into breeding pools from which cultivars are developed by selfing and selection of desired phenotypes or through the dihaploid breeding method followed by the selection of desired phenotypes. The new cultivars are evaluated to determine which have commercial potential.

Choice of breeding or selection methods depends on the mode of plant reproduction, the heritability of the trait(s) being improved, and the type of cultivar used commercially (e.g., F₁ hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, recurrent selection, and backcross breeding.

### A. Pedigree Selection

Pedigree breeding is used commonly for the improvement of self-pollinating crops or inbred lines of cross-pollinating crops. Two parents possessing favorable, complementary traits are crossed to produce an F₁. An F₂ population is produced by selfing one or several F₁s or by intercrossing two F₁s (sib mating). The dihaploid breeding method could also be used. Selection of the best individuals is usually begun in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release of new cultivars. Similarly, the development of new cultivars through the dihaploid system requires the selection of the cultivars followed by two to five years of testing in replicated plots.

### B. Backcross Breeding

Backcross breeding has been used to transfer genes for a simply inherited, highly heritable trait into a desirable homozygous cultivar or inbred line which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of recurrent parent and the trait of interest from the donor parent are selected and repeatedly crossed (backcrossed) to the recurrent parent. The resulting plant is expected to have the attributes of the recurrent parent (e.g., cultivar) and the desirable trait transferred from the donor parent.

When the term cultivar is used in the context of the present disclosure, this also includes any cultivar plant where one or more desired trait has been introduced through backcrossing methods, whether such trait is a naturally occurring spontaneous mutation(s), an induced mutation(s), or a gene or a nucleotide sequence modified by the use of New Breeding Techniques. Backcrossing methods can be used with the present disclosure to improve or introduce one or more characteristic into the cultivar of the present disclosure. The term “backcrossing” as used herein refers to the repeated crossing of a hybrid progeny back to the recurrent parent, i.e., backcrossing one, two, three, four, five, six, seven, eight, nine, or more times to the recurrent parent. The parental cultivar plant which contributes the gene or the genes for the desired characteristic is termed the nonrecurrent or donor parent. This terminology refers to the fact that the nonrecurrent parent is used one time in the backcross protocol and therefore does not recur. The parental cultivar to which the gene or genes from the nonrecurrent parent are transferred is known as the recurrent parent as it is used for several rounds in the backcrossing protocol.

### C. Open-Pollinated Populations

The improvement of open-pollinated populations of such crops as rye, maize and sugar beets, herbage grasses, legumes such as alfalfa and clover, and tropical tree crops such as cacao, coconuts, oil palm and some rubber, depends essentially upon changing gene-frequencies towards fixation of favorable alleles while maintaining a high (but far from maximal) degree of heterozygosity.

There are basically two primary methods of open-pollinated population improvement.

First, there is the situation in which a population is changed en masse by a chosen selection procedure. The outcome is an improved population that is indefinitely propagable by random-mating within itself in isolation.

Second, the synthetic variety attains the same end result as population improvement, but is not itself propagable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988).

### A) Mass Selection

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued. In mass selection, desirable individual plants are chosen, harvested, and the seed composited without progeny testing to produce the following generation. Since selection is based on the maternal parent only, and there is no control over pollination, mass selection amounts to a form of random mating with selection. As stated above, the purpose of mass selection is to increase the proportion of superior genotypes in the population.

### B) Synthetics

A synthetic variety is produced by intercrossing a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or topcrosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous.

Whether a synthetic can go straight from the parental seed production plot to the farmer or must first undergo one or more cycles of multiplication depends on seed production and the scale of demand for seed. In practice, grasses and clovers are generally multiplied once or twice and are thus considerably removed from the original synthetic.

While mass selection is sometimes used, progeny testing is generally preferred for polycrosses, because of their operational simplicity and obvious relevance to the objective, namely exploitation of general combining ability in a synthetic.

The number of parental lines or clones that enters a synthetic varies widely. In practice, numbers of parental lines range from 10 to several hundred, with 100-200 being the average. Broad based synthetics formed from 100 or more clones would be expected to be more stable during seed multiplication than narrow based synthetics.

### D. Hybrids

A hybrid is an individual plant resulting from a cross between parents of differing genotypes. Commercial hybrids are now used extensively in many crops, including corn (maize), sorghum, sugarbeet. sunflower and broccoli as well as leafy vegetables such as lettuce. Hybrids can be formed in a number of different ways, including by crossing two parents directly (single cross hybrids), by crossing a single cross hybrid with another parent (three-way or triple cross hybrids), or by crossing two different hybrids (four-way or double cross hybrids).

Strictly speaking, most individuals in an out breeding (i.e., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

Hybrid commercial seeds can be produced by insect pollination.

Once the inbreds that give the best hybrid performance have been identified, the hybrid seed can be reproduced indefinitely as long as the homogeneity of the inbred parent is maintained. A single-cross hybrid is produced when two inbred lines are crossed to produce the F1 progeny. A double-cross hybrid is produced from four inbred lines crossed in pairs (A×B and C×D) and then the two F1 hybrids are crossed again (A×B)×(C×D). Much of the hybrid vigor and uniformity exhibited by F1 hybrids is lost in the next generation (F2). Consequently, seed from F2 hybrid varieties is not used for planting stock.

### E. Bulk Segregation Analysis (BSA)

BSA, a.k.a. bulked segregation analysis, or bulk segregant analysis, is a method described by Michelmore et al. (Michelmore et al., 1991. Identification of markers linked to disease-resistance genes by bulked segregant analysis: a rapid method to detect markers in specific genomic regions by using segregating populations. Proceedings of the National Academy of Sciences. USA. 99:9828-9832) and Quarrie et al. (Quarrie et al., 1999, Journal of Experimental Botany, 50(337):1299-1306).

For BSA of a trait of interest, parental lines with certain different phenotypes are chosen and crossed to generate F2, doubled haploid or recombinant inbred populations with QTL analysis. The population is then phenotyped to identify individual plants or lines having high or low expression of the trait. Two DNA bulks are prepared, one from the individuals having one phenotype (e.g., resistant to virus), and the other from the individuals having reversed phenotype (e.g., susceptible to virus), and analyzed for allele frequency with molecular markers. Only a few individuals are required in each bulk (e.g., 10 plants each) if the markers are dominant (e.g., RAPDs). More individuals are needed when markers are co-dominant (e.g., RFLPs, SNPs or SSRs). Markers linked to the phenotype can be identified and used for breeding or QTL mapping.

### F. Hand-Pollination Method

Hand pollination describes the crossing of plants via the deliberate fertilization of female ovules with pollen from a desired male parent plant. In some embodiments the donor or recipient female parent and the donor or recipient male parent line are planted in the same field. In some embodiments the donor or recipient female parent and the donor or recipient male parent line are planted in the same greenhouse. The inbred male parent can be planted earlier than the female parent to ensure adequate pollen supply at the pollination time. In some embodiments, the male parent and female parent can be planted at a ratio of 1 male parent to 4-10 female parents. The male parent may be planted at the top of the field for efficient male flower collection during pollination. Pollination is started when the female parent flower is ready to be fertilized. Female flower buds that are ready to open in the following days are identified, covered with paper cups or small paper bags that prevent bee or any other insect from visiting the female flowers, and marked with any kind of material that can be easily seen the next morning. In some embodiments, this process is best done in the afternoon. The male flowers of the male parent are collected in the early morning before they are open and visited by pollinating insects. The covered female flowers of the female parent, which have opened, are un-covered and pollinated with the collected fresh male flowers of the male parent, starting as soon as the male flower sheds pollen. The pollinated female flowers are again covered after pollination to prevent bees and any other insects visit. The pollinated female flowers are also marked. The marked flowers are harvested. In some embodiments, the male pollen used for fertilization has been previously collected and stored.

### G. Bee-Pollination Method

Using the bee-pollination method, the parent plants are usually planted within close proximity. In some embodiments more female plants are planted to allow for a greater production of seed. Insects are placed in the field or greenhouses for transfer of pollen from the male parent to the female flowers of the female parent.

### H. Targeting Induced Local Lesions in Genomes (TILLING)

Breeding schemes of the present application can include crosses with TILLING® plant cultivars. TILLING® is a method in molecular biology that allows directed identification of mutations in a specific gene. TILLING® was introduced in 2000, using the model plant Arabidopsis thaliana. TILLING® has since been used as a reverse genetics method in other organisms such as zebrafish, corn, wheat, rice, soybean, tomato and lettuce.

The method combines a standard and efficient technique of mutagenesis with a chemical mutagen (e.g., Ethyl methanesulfonate (EMS)) with a sensitive DNA screening-technique that identifies single base mutations (also called point mutations) in a target gene. EcoTILLING is a method that uses TILLING® techniques to look for natural mutations in individuals, usually for population genetics analysis (see Comai, et al., 2003 The Plant Journal 37, 778-786; Gilchrist et al. 2006 Mol. Ecol. 15, 1367-1378; Mejlhede et al. 2006 Plant Breeding 125, 461-467; Nicto et al. 2007 BMC Plant Biology 7, 34-42. each of which is incorporated by reference hereby for all purposes). DEcoTILLING is a modification of TILLING® and EcoTILLING which uses an inexpensive method to identify fragments (Garvin et al., 2007, DEco-TILLING: An inexpensive method for SNP discovery that reduces ascertainment bias. Molecular Ecology Notes 7, 735-746).

The TILLING® method relies on the formation of heteroduplexes that are formed when multiple alleles (which could be from a heterozygote or a pool of multiple homozygotes and heterozygotes) are amplified in a PCR, heated, and then slowly cooled. As DNA bases are not pairing at the mismatch of the two DNA strands (the induced mutation in TILLING® or the natural mutation or SNP in EcoTILLING), they provoke a shape change in the double strand DNA fragment which is then cleaved by single stranded nucleases. The products are then separated by size on several different platforms.

Several TILLING® centers exists over the world that focus on agriculturally important species: UC Davis (USA), focusing on Rice; Purdue University (USA), focusing on Maize; University of British Columbia (CA), focusing on Brassica napus; John Innes Centre (UK), focusing on Brassica rapa; Fred Hutchinson Cancer Research, focusing on Arabidopsis; Southern Illinois University (USA), focusing on Soybean; John Innes Centre (UK), focusing on Lotus and Medicago; and INRA (France), focusing on Pea and Tomato.

More detailed description on methods and compositions on TILLING® can be found in U.S. Pat. No. 5,994,075, US 2004/0053236 A1, WO 2005/055704, and WO 2005/048692, each of which is hereby incorporated by reference for all purposes.

Thus in some embodiments, the breeding methods of the present disclosure include breeding with one or more TILLING plant lines with one or more identified mutations.

### I. Mutation Breeding

Mutation breeding is another method of introducing new variation and subsequent traits into plants. Mutations that occur spontaneously or are artificially induced can be useful sources of variability for a plant breeder. The goal of artificial mutagenesis is to increase the rate of mutation for a desired characteristic. Mutation rates can be increased by many different means or mutating agents including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in W. R. Fehr, 1993. Principles of Cultivar Development, Macmillan Publishing Co.

New breeding techniques such as the ones involving the uses of engineered nuclease to enhance the efficacy and precision of gene editing in combination with oligonucleotides including, but not limited to Zinc Finger Nucleases (ZFN), TAL effector nucleases (TALENs) and clustered regularly interspaced short palindromic repeats (CRISPR)-associated endonuclease Cas9 (CRISPR-Cas9) using such as Cas9, Cas12a/Cpf1, Cas13/C2c2, CasX and CasY or oligonucleotide directed mutagenesis shall also be used to generate genetic variability and introduce new traits into varieties.

### Genome Editing by CRISPR

Using genome editing, DNA can be modified in a targeted way, providing new alternatives to develop novel traits in plants.

Genome editing by CRISPR, which stands for Clustered Regularly Interspaced Short Palindromic Repeats, is based on a natural immune process used by bacteria to defend themselves against invading viruses. In bacteria, the invading viral DNA is cut through use of a guide RNA (gRNA), or piece of RNA, and a CRISPR-associated protein (Cas). The last step of the bacterial immune process, when the gRNA is combined with Cas and cleaves the target DNA, has been adopted for genome editing in laboratories.

There are at least three main CRISPR system types (Type I, II, and III) and at least 10 distinct subtypes (Makarova, K. S., et. al., Nat Rev Microbiol. 2011 May 9; 9(6):467-477). Type I and III systems use Cas protein complexes and short guide polynucleotide sequences to target selected DNA regions. Type II systems rely on a single protein (e.g. Cas9) and the targeting guide polynucleotide, where a portion of the 5′ end of a guide sequence is complementary to a target nucleic acid. For more information on the CRISPR gene editing compositions and methods of the present disclosure, see U.S. Pat. Nos. 8,697,359; 8,889,418; 8,771,945; and 8,871,445, each of which is hereby incorporated in its entirety for all purposes.

CRISPR genome editing requires two components, a gRNA and a Cas enzyme. These components associate to form a ribonucleoprotein (RNP) complex, where after the gRNA can base pair with a complementary protospacer sequence (i.e. the target genomic sequence of about 20 bases in length) under the condition that a particular adjacent sequence, called a protospacer-adjacent motif (PAM), is present in the genome. The PAM is only a few bases long, and its sequence depends on the type of Cas enzyme used. Once the gRNA binds to the target DNA (protospacer), the Cas enzyme recognizes this complex and makes a precise cut at the target site.

Either Cas9 or Cas 12a (also called Cpf1) can be used to cleave target DNA, resulting in a Double Strand Break (DSB). Each Cas enzyme is directed by the gRNA to a user-specified cut site in the genome. Like Cas9 nucleases, Cas12a1 family members contain a RuvC-like endonuclease domain, but lack the second HNH endonuclease domain of Cas9. Cas 12a cleaves DNA in a staggered pattern in contrast to Cas9 which produces a blunt-end. Moreover, for cleavage Cas12a requires only one RNA rather than the two tracrRNA and crRNA needed by Cas9. For Cas9 as well as Cas 12a, the target sequence of the gRNAs must be next to a PAM sequence. In the case of Cas9, the PAM sequence corresponds to NGG, where N is any base. The gRNA will recognize and bind to 20 nucleotides on the DNA strand opposite from the NGG PAM site. For Cas12a, the PAM sequence is TTTV, where V can represent A, C, or G. Using Alt-R Cas12a Ultra from Integrated DNA Technologies, a TTTT PAM sequence may also work. The “V” of the TTTV is immediately adjacent to the base at the 5′ end of the non-targeted strand side of the protospacer element. The guide RNA for Cas12a is relatively short and is approximately 40 to 44 bases long.

The damage caused by the double strand break (DSB) will be repaired in eukaryotic cells, primarily by two pathways: Non-Homologous End-Joining (NHEJ) and Homology Directed Repair (HDR). The HDR mechanism requires the presence of a donor DNA template containing regions of homology to both sites of the DNA break. This donor DNA can carry specific mutations and has to be delivered simultaneously with a preassembled Cas RNP complex composed of Cas9 or Cas12a and synthetically produced gRNAs.

Targeted cleavage events induced by nucleases are used to introduce targeted mutations (deletions, substitutions and insertions) in genomic DNA sequences and as such, in some embodiments, targeted cleavage is used as an efficient tool for genome editing in plants to introduce a genetic construct of the present disclosure into a plant genome.

In some aspects, the present disclosure relates to a recombinant DNA construct comprising an expression cassette(s) encoding a site-specific nuclease and, optionally, any associated protein(s) to carry out genome modification. These nuclease-expressing cassette(s) may be present in the same molecule or vector as a donor template for templated editing or an expression cassette comprising nucleic acid sequence encoding genetic constructs as described herein or on a separate molecule or vector. Several methods for site-directed integration are known in the art involving different sequence-specific nucleases (or complexes of proteins or guide RNA or both) that cut the genomic DNA to produce a double strand break (DSB) or nick at a desired genomic site or locus. As understood in the art, during the process of repairing the DSB or nick introduced by the nuclease enzyme, the donor template DNA, transgene, or expression cassette may become integrated into the genome at the site of the DSB or nick. The presence of the homology arm(s) in the DNA to be integrated may promote the adoption and targeting of the insertion sequence into the plant genome during the repair process through homologous recombination, although an insertion event may occur through non-homologous end joining (NHEJ).

In some aspects, the endonuclease is selected from a meganuclease, a zinc-finger nuclease (ZFN), a transcription activator-like effector nucleases (TALEN), an Argonaute (non limiting examples of Argonaute proteins include Thermus thermophilus Argonaute (TtAgo), Pyrococcus furiosus Argonaute (PfAgo), Natronobacterium gregoryi Argonaute (NgAgo), an RNA-guided nuclease, such as a CRISPR associated nuclease (non-limiting examples of CRISPR associated nucleases include Cas1, Cas1B, Cas2, Cas3, Cas4, Cas5, Cas6, Cas7, Cas8, Cas9 (also known as Csn1 and Csx12), Cas12a (also known as Cpf1), Cas1O, Csy1, Csy2, Csy3, Csc1, Csc2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, Cpf1, CasX, CasY, homologs thereof, or modified versions thereof.

In some aspects, the site-specific genome modification enzyme is a recombinase. Non-limiting examples of recombinases include a tyrosine recombinase attached to a DNA recognition motif provided herein is selected from the group consisting of a Cre recombinase, a Gin recombinase, a Flp recombinase, and a Tnp1 recombinase. In some aspects, a Cre recombinase or a Gin recombinase provided herein is tethered to a zinc-finger DNA-binding domain, or a TALE DNA-binding domain, or a Cas9 nuclease. In another aspect, a serine recombinase attached to a DNA recognition motif provided herein is selected from the group consisting of a PhiC3 1 integrase, an R4 integrase, and a TP-901 integrase. In another aspect, a DNA transposase attached to a DNA binding domain provided herein is selected from the group consisting of a TALE-piggy Bac and TALE-Mutator.

In some aspects, plants comprising one or more of the genetic alterations described herein may be selfed or crossed to produce lines that are homozygous for one or more of the genetic alterations described herein. In some aspects, the genetic alterations described herein may be transferred or introgressed to other plant varieties through conventional breeding schemes.

In some embodiments, the disclosure provides a guide RNA suitable for use in the CRISPR-Cas based genome editing system taught herein. In some embodiments, the disclosure provides a donor DNA suitable for use in the CRISPR-Cas based genome editing system taught herein. In some embodiments, the guide RNA and donor template are provided in a ribonucleoprotein (RNP) complex. In some embodiments, the guide RNA and donor template are provided in a plasmid.

In some embodiments, a CRISPR-Cas genome editing system comprising: (a) a first expression construct comprising a target locus-specific guide RNA (gRNA) and a donor template; and (b) a second expression construct comprising a polynucleotide encoding a CRISPR-associated protein nuclease. The CRISPR-Cas based genome editing system comprises at least one gRNA, a donor template, PAM sequence, and CRISPR-associated nuclease selected from the group consisting of Cas9, Cas12, Cas13, CasX, and CasY.

In some embodiments, the genome editing method comprises the steps of: a) transfecting a protoplast with a genome editing system to generate a transfected protoplast, wherein the genome editing system comprises: i) a Cas enzyme; ii) at least one guide RNA (gRNA); and iii) at least one single-stranded donor DNA repair template designed to introduce a genetic construct of the disclosure; b) exposing the transfected protoplast to a selective pressure; c) selecting a protoplast comprising the genetic construct; and d) regenerating a plant from said selected protoplast to produce a plant with the genetic construct incorporated.

### J. Double Haploids and Chromosome Doubling

One way to obtain homozygous plants without the need to cross two parental lines followed by a long selection of the segregating progeny, and/or multiple backcrossings is to produce haploids and then double the chromosomes to form doubled haploids. Haploid plants can occur spontaneously, or may be artificially induced via chemical treatments or by crossing plants with inducer lines (Seymour et al. 2012, PNAS vol 109, pg 4227-4232; Zhang et al., 2008 Plant Cell Rep. December 27(12) 1851-60). The production of haploid progeny can occur via a variety of mechanisms which can affect the distribution of chromosomes during gamete formation. The chromosome complements of haploids sometimes double spontaneously to produce homozygous doubled haploids (DHs). Mixoploids, which are plants which contain cells having different ploidies, can sometimes arise and may represent plants that are undergoing chromosome doubling so as to spontaneously produce doubled haploid tissues, organs, shoots, floral parts or plants. Another common technique is to induce the formation of double haploid plants with a chromosome doubling treatment such as colchicine (El-Hennawy et al., 2011 Vol 56, issue 2 pg 63-72; Doubled Haploid Production in Crop Plants 2003 edited by Maluszynski ISBN 1-4020-1544-5). The production of doubled haploid plants yields highly uniform cultivars and is especially desirable as an alternative to sexual inbreeding of longer-generation crops. By producing doubled haploid progeny, the number of possible gene combinations for inherited traits is more manageable. Thus, an efficient doubled haploid technology can significantly reduce the time and the cost of inbred and cultivar development.

### K. Protoplast Fusion

In another method for breeding plants, protoplast fusion can also be used for the transfer of trait-conferring genomic material from a donor plant to a recipient plant. Protoplast fusion is an induced or spontaneous union, such as a somatic hybridization, between two or more protoplasts (cells of which the cell walls are removed by enzymatic treatment) to produce a single bi- or multi-nucleate cell. The fused cell that may even be obtained with plant species that cannot be interbred in nature is tissue cultured into a hybrid plant exhibiting the desirable combination of traits.

### L. Embryo Rescue

Alternatively, embryo rescue may be employed in the transfer of resistance-conferring genomic material from a donor plant to a recipient plant. Embryo rescue can be used as a procedure to isolate embryos from crosses to rapidly move to the next generation of backcrossing or selfing or wherein plants fail to produce viable seed. In this process, the fertilized ovary or immature seed of a plant is tissue cultured to create new plants (see Pierik, 1999, In Vitro Culture of Higher Plants, Springer, ISBN 079235267, 9780792352679, which is incorporated herein by reference in its entirety).

### Breeding Evaluation

Each breeding program can include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.).

Promising advanced breeding lines are thoroughly tested per se and in hybrid combination and compared to appropriate standards in environments representative of the commercial target area(s). The best lines are candidates for use as parents in new commercial cultivars; those still deficient in a few traits may be used as parents to produce new populations for further selection or in a backcross program to improve the parent lines for a specific trait.

In some embodiments, the plants are selected on the basis of one or more phenotypic traits. Skilled persons will readily appreciate that such traits include any observable characteristic of the plant, including for example growth rate, vigor, plant health, maturity, plant height, leaf coverage, weight, total yield, color, taste, sugar levels, aroma, smell, changes in the production of one or more compounds by the plant (including for example, metabolites, proteins, drugs, carbohydrates, oils, and any other compounds).

A most difficult task is the identification of individuals that are genetically superior, because for most traits the true genotypic value is masked by other confounding plant traits or environmental factors. One method of identifying a superior plant is to observe its performance relative to other experimental plants and to a widely grown standard cultivar. If a single observation is inconclusive, replicated observations provide a better estimate of its genetic worth.

It should be appreciated that in certain embodiments, plants may be selected based on the absence, suppression or inhibition of a certain feature or trait (such as an undesirable feature or trait) as opposed to the presence of a certain feature or trait (such as a desirable feature or trait).

Selecting plants based on genotypic information is also envisaged (for example, including the pattern of plant gene expression, genotype, or presence of genetic markers). Where the presence of one or more genetic marker is assessed, the one or more marker may already be known and/or associated with a particular characteristic of a plant; for example, a marker or markers may be associated with an increased growth rate or metabolite profile. This information could be used in combination with assessment based on other characteristics in a method of the disclosure to select for a combination of different plant characteristics that may be desirable. Such techniques may be used to identify novel quantitative trait loci (QTLs). By way of example, plants may be selected based on growth rate, size (including but not limited to weight, height, leaf size, stem size, branching pattern, or the size of any part of the plant), general health, survival, tolerance to adverse physical environments and/or any other characteristic, as described herein before.

Further non-limiting examples include selecting plants based on: speed of seed germination; quantity of biomass produced; increased root, and/or leaf/shoot growth that leads to an increased yield (herbage or grain or fiber or oil) or biomass production; effects on plant growth that results in an increased seed yield for a crop; effects on plant growth which result in an increased yield; effects on plant growth that lead to an increased resistance or tolerance to disease including fungal, viral or bacterial diseases, to mycoplasma, or to pests such as insects, mites or nematodes in which damage is measured by decreased foliar symptoms such as the incidence of bacterial or fungal lesions, or area of damaged foliage or reduction in the numbers of nematode cysts or galls on plant roots, or improvements in plant yield in the presence of such plant pests and diseases; effects on plant growth that lead to increased metabolite yields; effects on plant growth that lead to improved aesthetic appeal which may be particularly important in plants grown for their form, color or taste, for example the color intensity of leaves, or the taste of said leaves.

### Molecular Breeding Evaluation Techniques

Selection of plants based on phenotypic or genotypic information may be performed using techniques such as, but not limited to: high through-put screening of chemical components of plant origin, sequencing techniques including high through-put sequencing of genetic material, differential display techniques (including DDRT-PCR, and DD-PCR), nucleic acid microarray techniques, RNA-seq (Transcriptome Sequencing), qRTPCR (quantitative real time PCR).

In one embodiment, the evaluating step of a plant breeding program involves the identification of desirable traits in progeny plants. Progeny plants can be grown in, or exposed to conditions designed to emphasize a particular trait (e.g. drought conditions for drought tolerance, lower temperatures for freezing tolerant traits). Progeny plants with the highest scores for a particular trait may be used for subsequent breeding steps.

In some embodiments, plants selected from the evaluation step can exhibit a 1%, 5%, 10%, 15%, 20%, 25%, 30%, 35%, 40%, 45%, 50%, 55%, 60%, 65%, 70%, 75%, 80%, 85%, 90%, 95%, 100%, 120% or more improvement in a particular plant trait compared to a control plant.

In other embodiments, the evaluating step of plant breeding comprises one or more molecular biological tests for genes or other markers. For example, the molecular biological test can involve probe hybridization and/or amplification of nucleic acid (e.g., measuring nucleic acid density by Northern or Southern hybridization, PCR) and/or immunological detection (e.g., measuring protein density, such as precipitation and agglutination tests, ELISA (e.g., Lateral Flow test or DAS-ELISA). Western blot, Radioimmune Assay (RIA), immune labeling, immunosorbent electron microscopy (ISEM), and/or dot blot).

The procedure to perform a nucleic acid hybridization, an amplification of nucleic acid (e.g., PCR, RT-PCR) or an immunological detection (e.g., precipitation and agglutination tests, ELISA (e.g., Lateral Flow test or DAS-ELISA), Western blot, RIA, immunogold or immunofluorescent labeling, immunosorbent electron microscopy (ISEM), and/or dot blot tests) are performed as described elsewhere herein and well-known by one skilled in the art.

In one embodiment, the evaluating step comprises PCR (semi-quantitative or quantitative), wherein primers are used to amplify one or more nucleic acid sequences of a desirable gene, or a nucleic acid associated with said gene, or QTL or a desirable trait (e.g., a co-segregating nucleic acid, or other marker).

In another embodiment, the evaluating step comprises immunological detection (e.g., precipitation and agglutination tests, ELISA (e.g., Lateral Flow test or DAS-ELISA), Western blot, RIA, immuno labeling (gold, fluorescent, or other detectable marker), immunosorbent electron microscopy (ISEM), and/or dot blot), wherein one or more gene or marker-specific antibodies are used to detect one or more desirable proteins. In one embodiment, said specific antibody is selected from the group consisting of polyclonal antibodies, monoclonal antibodies, antibody fragments, and combination thereof.

Reverse Transcription Polymerase Chain Reaction (RT-PCR) can be utilized in the present disclosure to determine expression of a gene to assist during the selection step of a breeding scheme. It is a variant of polymerase chain reaction (PCR), a laboratory technique commonly used in molecular biology to generate many copies of a DNA sequence, a process termed “amplification”. In RT-PCR, however, RNA strand is first reverse transcribed into its DNA complement (complementary DNA, or cDNA) using the enzyme reverse transcriptase, and the resulting cDNA is amplified using traditional or real-time PCR.

RT-PCR utilizes a pair of primers, which are complementary to a defined sequence on each of the two strands of the cDNA. These primers are then extended by a DNA polymerase and a copy of the strand is made after each cycle, leading to logarithmic amplification.

RT-PCR includes three major steps. The first step is the reverse transcription (RT) where RNA is reverse transcribed to cDNA using a reverse transcriptase and primers. This step is very important in order to allow the performance of PCR since DNA polymerase can act only on DNA templates. The RT step can be performed either in the same tube with PCR (one-step PCR) or in a separate one (two-step PCR) using a temperature between 40° C. and 60° C. depending on the properties of the reverse transcriptase used.

The next step involves the denaturation of the dsDNA at 95° C. so that the two strands separate and the primers can bind again at lower temperatures and begin a new chain reaction. Then, the temperature is decreased until it reaches the annealing temperature which can vary depending on the set of primers used, their concentration, the probe and its concentration (if used), and the cation concentration. The main consideration, of course, when choosing the optimal annealing temperature is the melting temperature (Tm) of the primers and probes (if used). The annealing temperature chosen for a PCR depends directly on length and composition of the primers. This is the result of the difference of hydrogen bonds between A-T (2 bonds) and G-C (3 bonds). An annealing temperature about 5 degrees below the lowest Tm of the pair of primers is usually used.

The final step of PCR amplification is the DNA extension from the primers which is done by the thermostable Taq DNA polymerase usually at 72° C. which is the optimal temperature for the polymerase to work. The length of the incubation at each temperature, the temperature alterations and the number of cycles are controlled by a programmable thermal cycler. The analysis of the PCR products depends on the type of PCR applied. If a conventional PCR is used, the PCR product is detected using for example agarose gel electrophoresis or other polymer gel like polyacrylamide gels and ethidium bromide (or other nucleic acid staining).

Conventional RT-PCR is a time-consuming technique with important limitations when compared to real time PCR techniques. This, combined with the fact that ethidium bromide has low sensitivity, yields results that are not always reliable. Moreover, there is an increased cross-contamination risk of the samples since detection of the PCR product requires the post-amplification processing of the samples. Furthermore, the specificity of the assay is mainly determined by the primers, which can give false-positive results. However, the most important issue concerning conventional RT-PCR is the fact that it is a semi or even a low quantitative technique, where the amplicon can be visualized only after the amplification ends.

Real time RT-PCR provides a method where the amplicons can be visualized as the amplification progresses using a fluorescent reporter molecule. There are three major kinds of fluorescent reporters used in real time RT-PCR, general nonspecific DNA Binding Dyes such as SYBR Green I, TaqMan Probes and Molecular Beacons (including Scorpions).

The real time PCR thermal cycler has a fluorescence detection threshold, below which it cannot discriminate the difference between amplification generated signal and background noise. On the other hand, the fluorescence increases as the amplification progresses and the instrument performs data acquisition during the annealing step of each cycle. The number of amplicons will reach the detection baseline after a specific cycle, which depends on the initial concentration of the target DNA sequence. The cycle at which the instrument can discriminate the amplification generated fluorescence from the background noise is called the threshold cycle (Ct). The higher is the initial DNA concentration, the lower its Ct will be.

Other forms of nucleic acid detection can include next generation sequencing methods such as DNA SEQ or RNA SEQ using any known sequencing platform including, but not limited to: Roche 454, Solexa Genome Analyzer, AB SOLID, Illumina GA/HiSe,. Ion PGM, Mi Seq, among others (Liu et al., 2012 Journal of Biomedicine and Biotechnology Volume 2012 ID 251364; Franca et al., 2002 Quarterly Reviews of Biophysics 35 pg 169-200; Mardis 2008 Genomics and Human Genetics vol 9 pg 387-402).

In other embodiments, nucleic acids may be detected with other high throughput hybridization technologies including microarrays, gene chips, LNA probes, nanoStrings, and fluorescence polarization detection among others.

In some embodiments, detection of markers can be achieved at an early stage of plant growth by harvesting a small tissue sample (e.g., branch, or leaf disk). This approach is preferable when working with large populations as it allows breeders to weed out undesirable progeny at an early stage and conserve growth space and resources for progeny which show more promise. In some embodiments the detection of markers is automated, such that the detection and storage of marker data is handled by a machine. Recent advances in robotics have also led to full service analysis tools capable of handling nucleic acid/protein marker extractions, detection, storage and analysis.

### Quantitative Trait Loci

Breeding schemes of the present application can include crosses between donor and recipient plants. In some embodiments, said donor plants contain a gene or genes of interest which may confer the plant with a desirable phenotype. The recipient line can be an elite line or cultivar having certain favorite traits for commercial production. In one embodiment, the elite line may contain other genes that also impart said line with the desired phenotype. When crossed together, the donor and recipient plant may create a progeny plant with combined desirable loci which may provide quantitatively additive effect of a particular characteristic. In that case, QTL mapping can be involved to facilitate the breeding process.

A QTL (quantitative trait locus) mapping can be applied to determine the parts of the donor plant's genome conferring the desirable phenotype, and facilitate the breeding methods. Inheritance of quantitative traits or polygenic inheritance refers to the inheritance of a phenotypic characteristic that varies in degree and can be attributed to the interactions between two or more genes and their environment. Though not necessarily genes themselves, quantitative trait loci (QTLs) are stretches of DNA that are closely linked to the genes that underlie the trait in question. QTLs can be molecularly identified to help map regions of the genome that contain genes involved in specifying a quantitative trait. This can be an early step in identifying and sequencing these genes.

Typically, QTLs underlie continuous traits (those traits that vary continuously, e.g. yield, height, level of resistance to virus, etc.) as opposed to discrete traits (traits that have two or several character values, e.g. smooth vs. wrinkled peas used by Mendel in his experiments). Moreover, a single phenotypic trait is usually determined by many genes. Consequently, many QTLs are associated with a single trait.

A quantitative trait locus (QTL) is a region of DNA that is associated with a particular phenotypic trait. Knowing the number of QTLs that explains variation in the phenotypic trait tells about the genetic architecture of a trait. It may tell that a trait is controlled by many genes of small effect, or by a few genes of large effect or by a several genes of small effect and few genes of larger effect.

Another use of QTLs is to identify candidate genes underlying a trait. Once a region of DNA is identified as contributing to a phenotype, it can be sequenced. The DNA sequence of any genes in this region can then be compared to a database of DNA for genes whose function is already known.

In a recent development, classical QTL analyses are combined with gene expression profiling i.e. by DNA microarrays. Such expression QTLs (e-QTLs) describes cis- and trans-controlling elements for the expression of often disease-associated genes. Observed epistatic effects have been found beneficial to identify the gene responsible by a cross-validation of genes within the interacting loci with metabolic pathway and scientific literature databases.

QTL mapping is the statistical study of the alleles that occur in a locus and the phenotypes (physical forms or traits) that they produce (see, Meksem and Kahl, The handbook of plant genome mapping: genetic and physical mapping, 2005, Wiley-VCH, ISBN 3527311165. 9783527311163). Because most traits of interest are governed by more than one gene, defining and studying the entire locus of genes related to a trait gives hope of understanding what effect the genotype of an individual might have in the real world.

Statistical analysis is required to demonstrate that different genes interact with one another and to determine whether they produce a significant effect on the phenotype. QTLs identify a particular region of the genome as containing one or several genes, i.e. a cluster of genes that are associated with the trait being assayed or measured. They are shown as intervals across a chromosome, where the probability of association is plotted for each marker used in the mapping experiment.

To begin, a set of genetic markers must be developed for the species in question. A marker is an identifiable region of variable DNA. Biologists are interested in understanding the genetic basis of phenotypes (physical traits). The aim is to find a marker that is significantly more likely to co-occur with the trait than expected by chance, that is, a marker that has a statistical association with the trait. Ideally. they would be able to find the specific gene or genes in question, but this is a long and difficult undertaking. Instead, they can more readily find regions of DNA that are very close to the genes in question. When a QTL is found, it is often not the actual gene underlying the phenotypic trait, but rather a region of DNA that is closely linked with the gene.

For organisms whose genomes are known, one might now try to exclude genes in the identified region whose function is known with some certainty not to be connected with the trait in question. If the genome is not available, it may be an option to sequence the identified region and determine the putative functions of genes by their similarity to genes with known function, usually in other genomes. This can be done using BLAST, an online tool that allows users to enter a primary sequence and search for similar sequences within the BLAST database of genes from various organisms.

Another interest of statistical geneticists using QTL mapping is to determine the complexity of the genetic architecture underlying a phenotypic trait. For example, they may be interested in knowing whether a phenotype is shaped by many independent loci, or by a few loci, and how those loci interact. This can provide information on how the phenotype may be evolving.

Molecular markers are used for the visualization of differences in nucleic acid sequences. This visualization is possible due to DNA-DNA hybridization techniques (RFLP) and/or due to techniques using the polymerase chain reaction (e.g. STS, SNPs, microsatellites. AFLP). All differences between two parental genotypes will segregate in a mapping population based on the cross of these parental genotypes. The segregation of the different markers may be compared and recombination frequencies can be calculated. The recombination frequencies of molecular markers on different chromosomes are generally 50%. Between molecular markers located on the same chromosome the recombination frequency depends on the distance between the markers. A low recombination frequency usually corresponds to a low distance between markers on a chromosome. Comparing all recombination frequencies will result in the most logical order of the molecular markers on the chromosomes. This most logical order can be depicted in a linkage map (Paterson, 1996, Genome Mapping in Plants, R. G. Landes, Austin.). A group of adjacent or contiguous markers on the linkage map that is associated to a reduced disease incidence and/or a reduced lesion growth rate pinpoints the position of a QTL.

The nucleic acid sequence of a QTL may be determined by methods known to the skilled person. For instance, a nucleic acid sequence comprising said QTL or a resistance-conferring part thereof may be isolated from a donor plant by fragmenting the genome of said plant and selecting those fragments harboring one or more markers indicative of said QTL. Subsequently, or alternatively, the marker sequences (or parts thereof) indicative of said QTL may be used as (PCR) amplification primers, in order to amplify a nucleic acid sequence comprising said QTL from a genomic nucleic acid sample or a genome fragment obtained from said plant. The amplified sequence may then be purified in order to obtain the isolated QTL. The nucleotide sequence of the QTL, and/or of any additional markers comprised therein, may then be obtained by standard sequencing methods.

One or more such QTLs associated with a desirable trait in a donor plant can be transferred to a recipient plant to incorporate the desirable trait into progeny plants by transferring and/or breeding methods.

In one embodiment, an advanced backcross QTL analysis (AB-QTL) is used to discover the nucleotide sequence or the QTLs responsible for the resistance of a plant. Such method was proposed by Tanksley and Nelson in 1996 (Tanksley and Nelson, 1996, Advanced backcross QTL analysis: a method for simultaneous discovery and transfer of valuable QTL from un-adapted germplasm into elite breeding lines. Theor Appl Genet 92:191-203) as a new breeding method that integrates the process of QTL discovery with variety development, by simultaneously identifying and transferring useful QTL alleles from un-adapted (e.g., land races, wild species) to elite germplasm, thus broadening the genetic diversity available for breeding. AB-QTL strategy was initially developed and tested in tomato, and has been adapted for use in other crops including rice, maize, wheat, pepper, barley, and bean. Once favorable QTL alleles are detected, only a few additional marker-assisted generations are required to generate near isogenic lines (NILs) or introgression lines (ILs) that can be field tested in order to confirm the QTL effect and subsequently used for variety development.

Isogenic lines in which favorable QTL alleles have been fixed can be generated by systematic backcrossing and introgressing of marker-defined donor segments in the recurrent parent background. These isogenic lines are referred to as near isogenic lines (NILs), introgression lines (ILs), backcross inbred lines (BILs), backcross recombinant inbred lines (BCRIL), recombinant chromosome substitution lines (RCSLs), chromosome segment substitution lines (CSSLs), and stepped aligned inbred recombinant strains (STAIRSs). An introgression line in plant molecular biology is a line of a crop species that contains genetic material derived from a similar species. ILs represent NILs with relatively large average introgression length, while BILs and BCRILs are backcross populations generally containing multiple donor introgressions per line. As used herein, the term “introgression lines or ILs” refers to plant lines containing a single marker defined homozygous donor segment, and the term “pre-ILs” refers to lines which still contain multiple homozygous and/or heterozygous donor segments.

To enhance the rate of progress of introgression breeding, a genetic infrastructure of exotic libraries can be developed. Such an exotic library comprises a set of introgression lines, each of which has a single, possibly homozygous, marker-defined chromosomal segment that originates from a donor exotic parent, in an otherwise homogenous elite genetic background, so that the entire donor genome would be represented in a set of introgression lines. A collection of such introgression lines is referred as libraries of introgression lines or IL libraries (ILLs). The lines of an ILL cover usually the complete genome of the donor, or the part of interest. Introgression lines allow the study of quantitative trait loci, but also the creation of new varieties by introducing exotic traits. High resolution mapping of QTL using ILLs enable breeders to assess whether the effect on the phenotype is due to a single QTL or to several tightly linked QTL affecting the same trait. In addition, sub-ILs can be developed to discover molecular markers which are more tightly linked to the QTL of interest, which can be used for marker-assisted breeding (MAB). Multiple introgression lines can be developed when the introgression of a single QTL is not sufficient to result in a substantial improvement in agriculturally important traits (Gur and Zamir, Unused natural variation can lift yield barriers in plant breeding, 2004, PLOS Biol.; 2(10):e245).

### Tissue Culture

As it is well known in the art, tissue culture of plants can be used for the in vitro regeneration of plants. Tissues cultures of various tissues of plants and regeneration of plants therefrom are well known and published. For example, reference may be had to Teng et al., HortScience, 27:9, 1030-1032 (1992), Teng et al., HortScience, 28:6, 669-671 (1993), Zhang et al., Journal of Genetics and Breeding. 46: 3, 287-290 (1992), Webb et al., Plant Cell Tissue and Organ Culture, 38: 1, 77-79 (1994). Curtis et al., Journal of Experimental Botany, 45: 279, 1441-1449 (1994), Nagata et al., Journal for the American Society for Horticultural Science, 125: 6, 669-672 (2000). It is clear from the literature that the state of the art is such that these methods of obtaining plants are routinely used and have a very high rate of success. Thus, another aspect of this disclosure is to provide cells which upon growth and differentiation produce plants having all the physiological and morphological characteristics of a plant disclosed herein.

As used herein, the term “tissue culture” indicates a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as embryos, pollens, flowers, seeds, leaves, stems, roots, root tips, anthers, pistils, meristematic cells, axillary buds, ovaries, seed coats, endosperms, hypocotyls, cotyledons and the like. Means for preparing and maintaining plant tissue culture are well known in the art. By way of example, a tissue culture comprising organs has been used to produce regenerated plants. U.S. Pat. Nos. 5,959,185, 5,973,234, and 5,977,445 describe certain techniques, the disclosures of which are incorporated herein by reference.

### Methods

The present disclosure provides methods for reducing silencing of a gene of interest. The methods comprise delivering the genetic constructs of the disclosure to a host plant cell. Means of delivery include any of those disclosed herein, e.g., as disclosed in the plant transformation and plant breeding sections.

In some embodiments, the expression of the gene of interest improves a growing parameter, production parameter, or biostimulant parameter of a host plant.

In some embodiments, the method increases a growing parameter of the host plant. A growing parameter is related to the growth of the host plant. Growing parameters include plant size, biomass (dry or wet), aerial biomass, height, number of branches, number of leaves, number of flowers, root biomass, number of roots, number of secondary roots, root volume, root length, and degree of inoculation by diazotrophic bacteria.

In some embodiments, the method increases a production parameter of the host plant. A production parameter is related to the plant part that is harvested from the plant for commercial purposes. Production parameters include, but are not limited to, yield, yield per plant, yield per area, harvested biomass, harvested weight, harvested volume, number of harvested plant parts, and size of harvested plant parts. In terms of the harvestable plant parts, production parameters include yield, weight, size, and number of harvestable plant parts. Harvestable plant parts include, for example, fruits, vegetables, roots, grains, tubers, leaves, flowers, seeds, and nuts. In some embodiments, e.g., for some grasses, lettuces, feed crops, and forage crops, a harvestable plant part is the entire aerial biomass of the plant. In some embodiments, the harvestable plant part is related to the intended use of the crop. For example, for oil crops, the harvestable plant parts are the components of the plant containing the oil to be harvested.

In some embodiments, the method increases a biochemical parameter of the host plant. Biochemical parameters include, but are not limited to, chlorophyl content, carotenoid content, micronutrient profile, and macronutrient profile. In some embodiments, the method increases the concentration of a chlorophyl, e.g., chlorophyl a or chlorophyl b. In some embodiments, the method increases the concentration of a carotenoid or improves the average carotenoid profile. In some embodiments, the method increases the micro and/or macro-nutrient profile of the harvested plant part, the plant leaves, or the plant roots. In some embodiments, the method increases the concentration of one or more micronutrients or one or more macronutrients in the roots, leaves, or fruits of the host plant. In some embodiments, the method increases the nitrogen content in the leaves of the host plant. Nitrogen stimulates plant growth and is directly related to the root system's ability to fix nitrogen and the host plant's nitrogen metabolism. In some embodiments, the method increases the concentration of magnesium, manganese, copper, or potassium in the roots. Manganese and Copper are highly effective micronutrients in plant resistance to diseases (Marschner, 2012). By affecting cell wall composition and lignin synthesis Mn and Cu suppress penetration of pathogens into plant tissue. Increases in chlorophyl content depend on Mg supply (Marschner, 2012). Plant Stem Growth is very sensitive to potassium concentration. Plant height increase can be related to potassium concentration in the root system. Potassium is also involved in tree growth and wood formation. In the cambial region and the xylem differentiation zone, a strong potassium demand has been shown. Differentiating xylem cells involved in wood formation represent a strong sink for potassium that provides the driving force for cell expansion (Langer et al., 2002; Plant Journal. 32:997-1009).

In some embodiments, the method improves a growing parameter, production parameter, or biochemical parameter compared to a control condition. In some embodiments, the method improves a parameter in terms of timing, i.e., the parameter is improved at a given time point compared to the control. For example, in some embodiments, the method may improve a growing parameter relative to a control early on, such as early flowering, faster maturation, increased height compared to control at the same time point.

In some embodiments, desirable traits that may be incorporated by this disclosure are improved resistance to different insects or viral, fungal, and bacterial pathogens.

In some embodiments, the methods of the disclosure improve production of a biomolecule in a host plant cell. In some embodiments, the biomolecule is a protein, nucleic acid, lipid, or carbohydrate.

### EXAMPLES

### Example 1: Genetic Constructs for Suppression of Plant Gene Silencing

### Construct Design

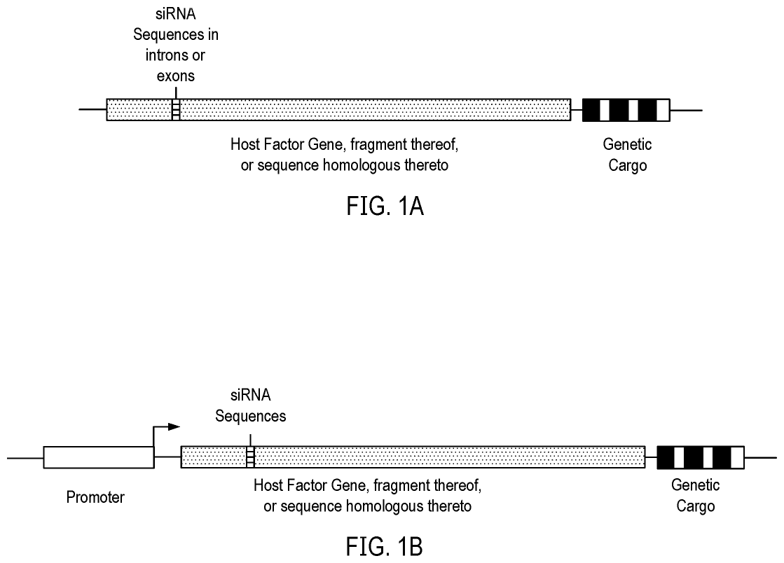

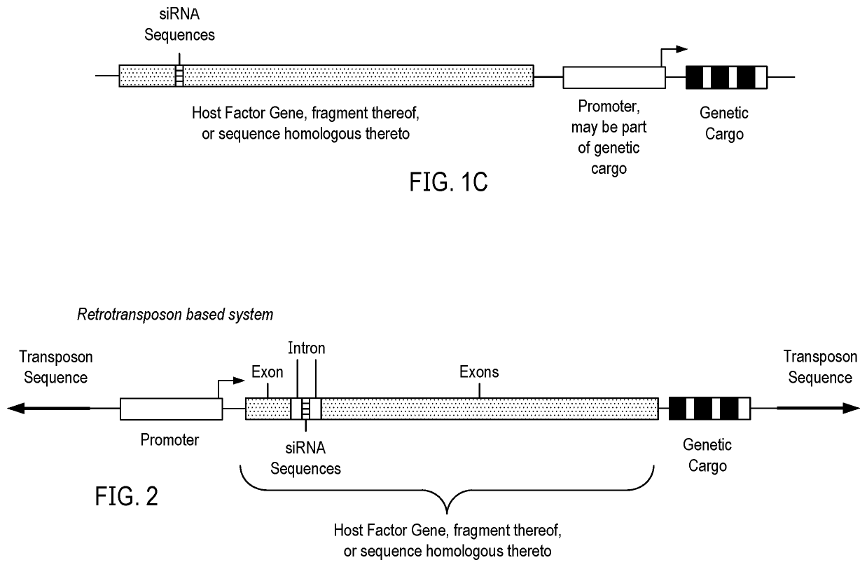

Illustrative genetic constructs of the disclosure are generated with a host factor sequence in proximity to a genetic cargo on the same polynucleotide (FIG. 1A). In some embodiments, the genetic construct comprises a promoter that acts on both the host factor sequence and on the genetic cargo (FIG. 1B). In some embodiments, the genetic construct comprises a promoter that acts on the genetic cargo or is comprised by the genetic cargo (FIG. 1C).

The host factor is involved in gene silencing. In some embodiments, the host factor is selected from DNA-directed RNA Polymerase V, DNA methyltransferase, dicer proteins, dicer-like proteins, argonaute proteins, and RNA-dependent RNA polymerases.

The genetic cargo comprises a gene of interest. The gene of interest is any transgene or endogenous gene for which expression or increased expression within the plant cell is desired.

The genetic construct is integrated into the plant genome by a method known in the art.

The host factor gene is related to regulation of invasive genetic elements in plants. Silencing of the host factor gene by siRNA-mediated cleavage or heterochromatin formation causes the plant cell to lose the ability to effectively silence the gene of interest. In this way, targeting the siRNA/DNA methylation pathways directly allows for interference with the very immune response used to silence the gene of interest.

### Example 2: Retrotransposon-Based Genetic Construct for Suppression of Plant Gene Silencing

### Construct Design

A retrotransposon-based vector for suppressing silencing of a gene of interest in plant cells is developed. The vector comprises a retrotransposon-based design with the genetic construct integrated into an intergenic region on the vector. The genetic construct comprises a promoter, a host factor sequence, and a genetic cargo. In this illustrative embodiment, the gene encoding the host factor comprises an intron comprising siRNA sequences. See FIG. 2.

The host factor is involved in gene silencing. In some embodiments, the host factor is selected from DNA-directed RNA Polymerase V, DNA methyltransferase, dicer proteins, dicer-like proteins, argonaute proteins, and RNA-dependent RNA polymerases.

The genetic cargo comprises a gene of interest, which is any transgene or endogenous gene for which expression or increased expression within the plant cell is desired.

The host factor gene is related to regulation of invasive genetic elements in plants. Silencing of the host factor gene by siRNA-mediated cleavage or heterochromatin formation causes the plant cell to lose the ability to effectively silence the gene of interest. In this way, targeting the siRNA/DNA methylation pathways directly allows for interference with the very immune response used to silence the gene of interest.

### Example 3: CRISPR-Based Genetic Construct for Suppression of Plant Gene Silencing

### Construct Design

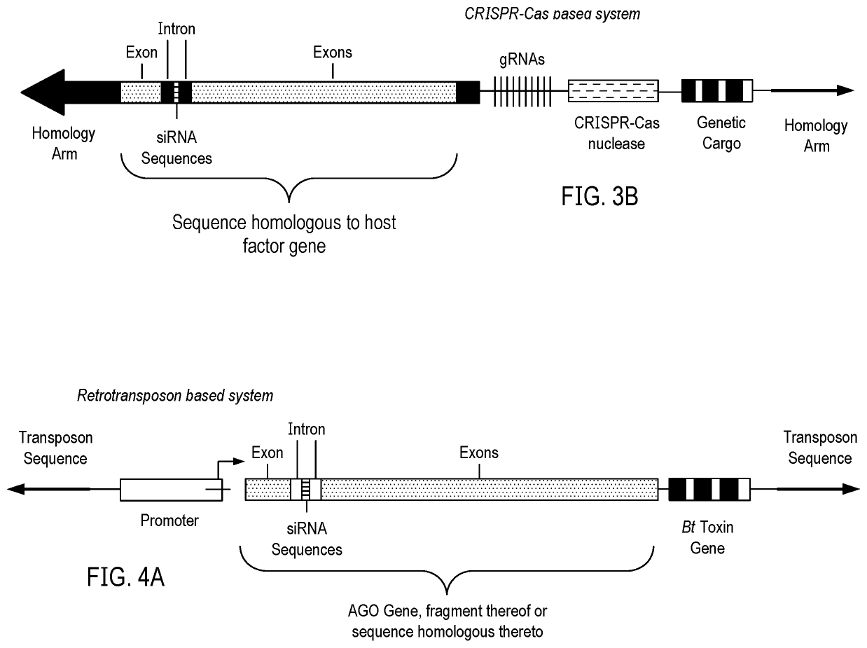

A CRISPR-based vector for suppressing silencing of a gene of interest in plant cells is developed. The vector comprises a promoter, a host factor sequence, and a genetic cargo. The host factor sequence comprises an intron comprising siRNA sequences. The vector comprises gRNA sequences, a CRISPR-Cas nuclease gene, and flanking homology arms. See FIG. 3A. The host factor is involved in gene silencing. In some embodiments, the host factor is selected from DNA-directed RNA Polymerase V. DNA methyltransferase, dicer proteins, dicer-like proteins, argonaute proteins, and RNA-dependent RNA polymerases.

In FIG. 3B, the homology arms are homologous to the host factor gene within the host plant, but with novel siRNA sequences incorporated. The host factor is required for fertility or survival.

In both FIG. 3A and FIG. 3B, the genetic cargo comprises a gene of interest that is any transgene or endogenous gene for which expression or increased expression within the plant cell is desired.

In FIG. 3A, the host factor is related to regulation of invasive genetic elements in plants. Silencing of the host factor gene by siRNA-mediated cleavage or heterochromatin formation causes the plant cell to lose the ability to effectively silence the gene of interest. In this way, targeting the siRNA/DNA methylation pathways directly allows for interference with the very immune response used to silence the gene of interest.

Using the construct of FIG. 3B, the host factor is integrated into the homology arms in order to target the desired host factor as the integration site for the genetic construct. Novel siRNA sequences are incorporated into these homology arms. NHEJ results in infertility or death, since the host factor function is impaired. HDR results in integration of the gene of interest linked to the host factor comprising novel siRNA sequences. Silencing of the host factor gene leads to death or infertility. In some embodiments, incorporation of the host factor into the homology arms limits the number of integration sites to the number of copies of the host factor within the genome. A host factor with repeated copies throughout the genome is selected to ensure multiple integrations sites. The use of multiple integration sites also makes it harder for drive resistance to develop via NHEJ. See also Noble et al., “Evolutionary dynamics of CRISPR gene drives,” Science Advances 2017; 3(4): e1601964.

### Example 4: Genetic Construct for Suppression of Transgenic Insecticidal Bt Protein Silencing

### Construct Design

Genetic constructs are developed according to Examples 1 and 2. The gene of interest encodes a Bacillus thuringiensis (Bt) insecticidal protein. The host factor gene is an Argonaute (AGO) gene.

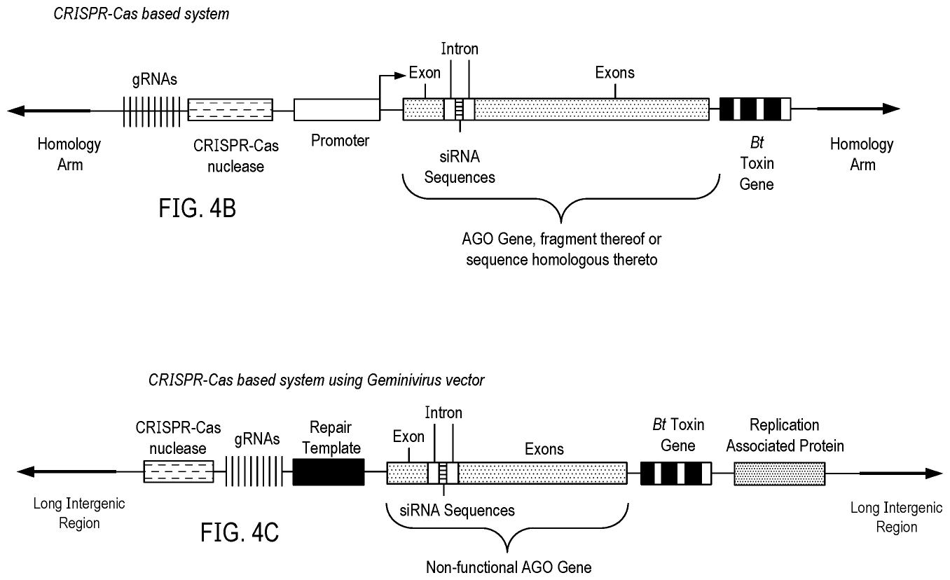

FIGS. 4A-C show illustrative genetic constructs comprising AGO host factor genes and genes of interest encoding Bt toxins. FIG. 4C shows a geminivirus vector comprising a CRISPR-based system comprising a genetic construct of the disclosure.

Use of these constructs results in suppression of silencing of Bt toxin gene in a host plant cell.

### Example 5: Genetic Construct for Suppression of Endogenous Plant Gene Silencing

### Construct Design

Genetic constructs are developed according to Examples 1 and 2. The gene of interest is an endogenous aldehyde dehydrogenase (ALDH) gene, which scavenges redundant aldehydes when the plant is exposed to stress. The ALDH gene is targeted for increased expression within the plant cell. The host factor gene encodes a dicer-like (DCL) protein.

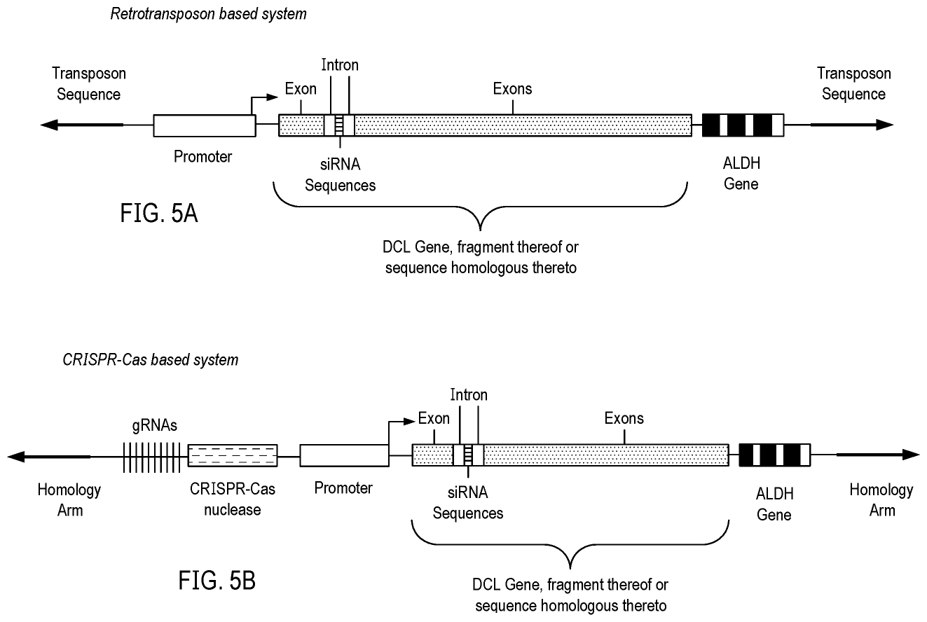

FIGS. 5A-B show illustrative genetic constructs comprising DCL host factor genes and endogenous ALDH genes of interest.

Use of these constructs results in increased expression and suppressed silencing of the ALDH gene, improving the host plant response to stress.

### Example 6: Genetic Construct for Suppression of Plant Gene Silencing Without siRNA Sequences

### Construct Design

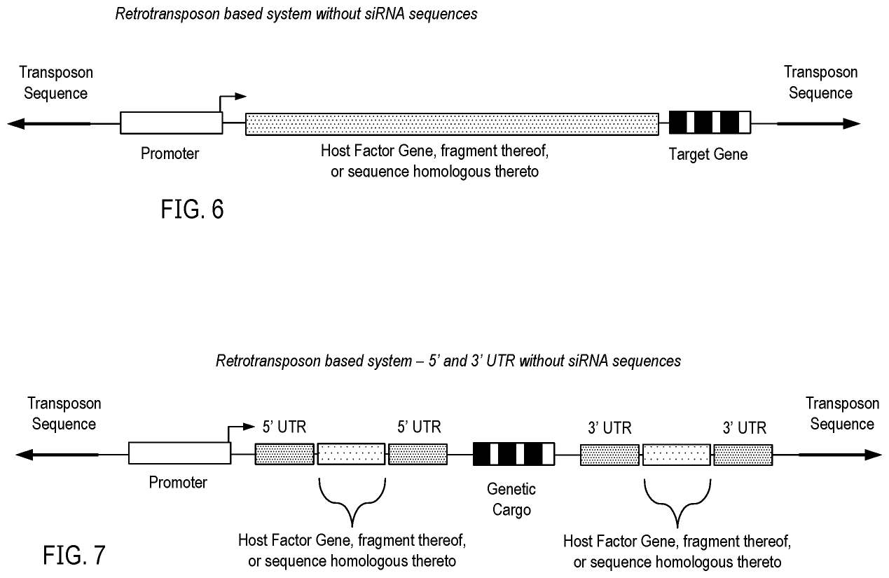

In some embodiments, a genetic construct is developed according to Example 1 or 2 without siRNA sequences. See an illustrative construct in FIG. 6. The host factor gene is necessary for survival or fertility or is a gene involved in gene silencing.

The proximity between the host factor gene and the gene of interest, in addition to their shared promoter, couples their silencing, such that silencing of either gene on the construct leads to silencing of both genes. Construct silencing thus leads to host factor gene silencing, resulting in deleterious host cell effects and/or suppression of the silencing pathways. As a result, silencing is suppressed, and gene of interest expression and/or sustained mobilization is improved.

### Example 7: Genetic Construct for Expression of a Cleavage-Resistant Proteins in Plants

### Construct Design

Human proteins, such as GLP-1, Exendin-4, collagen, retinol, FGF 1/2/9, TGF-β1, insulin, albumin, and heme, are modified to resist trypsin digestion and DPP-4 cleavage. For example, a DPPIV sensitive site in GLP-1 is mutated from Ala8 to Ser8 and two tryptic cleavage sites in GLP-1, Lys²⁶ and Lys³⁴, are mutated to Gln²⁶ and to Asp³⁴, respectively. See Sugita et al., FEBS Letters, 579(5) 14 1085-1088 (2005). The modified protein is then cloned into a 5× tandem repeat vector with furin cleavage sites between the different modified protein subunits to increase accumulation and prevent proteolytic degradation of single modified protein units. Each protein unit is linked with a transferrin peptide to enable transmucosal delivery. See Choi et al., Plant Biotechnology Journal. 12(4) 425-435 (2013). A hinge-furrin site allows for in vitro cleavage after uptake and reduces steric hindrance. See Boyhan and Daniell, Plant Biotechnology Journal, 9(5) 585-598 (2010). Alternatively, Cholera toxin B subunit may be use for in vitro cleavage.

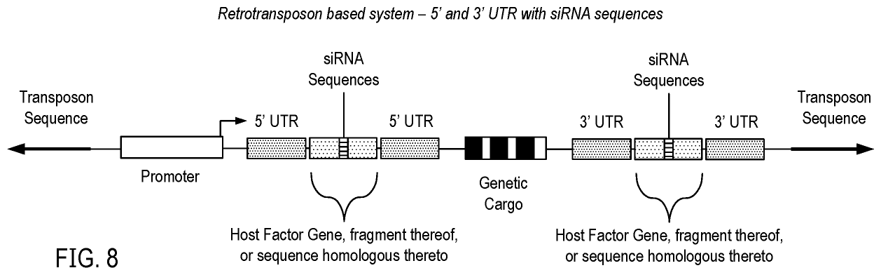

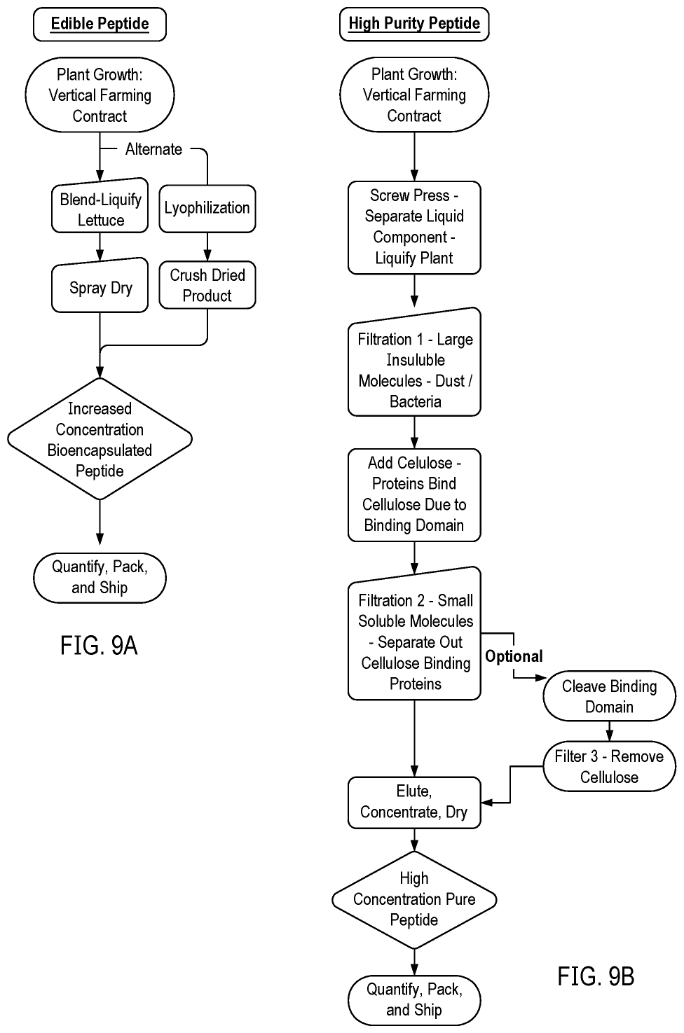

FIG. 7 shows an illustrative genetic construct comprising a retrotransposon-based system—5′ and 3′ UTR without siRNA sequences. FIG. 8 shows an illustrative genetic construct comprising a retrotransposon-based system—5′ and 3′ UTR with siRNA sequences. This cleavage-resistant proteins are also linked to eGFP with a P2A site for enzymatic cleavage in the plant as a marker. FIG. 9A-9B is a diagram that shows processing of the plants for peptide extraction at low (FIG. 9A) and high purities (FIG. 9B). See agfundernews.com/biobetter-bets-on-tobacco-to-bring-down-the-cost-of-growth-factors-for-cultivated-meat. Use of these constructs results in expression of cleavage-resistant proteins, such as GLP-1, Exendin-4, collagen, retinol, FGF 1/2/9. TGF-β1, insulin, albumin, and heme, in plants. The plants are harvested and the cleavage-resistant proteins are extracted at both low and high purities, as depicted in FIG. 9A-9B.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more.” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising. such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional. unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BB, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope, and concept of the invention as defined by the appended claims.

### NUMBERED EMBODIMENTS OF THE INVENTION

Notwithstanding the appended claims, the disclosure sets forth the following numbered embodiments:
1. A genetic construct comprising: (a) a plant host factor sequence operably linked to a genetic cargo sequence comprising a gene of interest; and (b) an siRNA sequence, wherein the siRNA sequence increases the likelihood that silencing of the genetic construct will lead to silencing of the plant host factor sequence.
2. The construct of embodiment 1, wherein the host factor sequence is a host factor gene, a fragment thereof, or a sequence homologous thereto.
3. The construct of embodiment 1 or 2, wherein the host factor is involved in host plant fecundity, survival, or silencing.
4. The construct of any one of embodiments 1-3, further comprising one or more host factor exon sequences at a 5′ and/or 3′ UTR with or without siRNA inclusions to direct silencing.
5. The construct of any one of embodiments 1-4, wherein the host factor is a DNA-directed RNA polymerase, a DNA methyltransferase, a dicer protein, a dicer-like protein, an argonaute protein, or an RNA-dependent RNA polymerase.
6. The construct of any one of embodiments 1-5, wherein the host factor is involved in host plant gene silencing via RNA-directed DNA methylation.
7. The construct of any one of embodiments 1-6, wherein the host factor is Argonaute 4 (AGO4), DNA-directed RNA polymerase V, or DNA methyltransferase.
8. The construct of any one of embodiments 1-7, wherein the gene of interest is a gene endogenous to the host plant cell.
9. The construct of any one of embodiments 1-8, wherein the gene of interest is a transgene.
10. The construct of any one of embodiments 1-9, wherein the gene of interest encodes a biocidal protein.
11. The construct of any one of embodiments 1-10, wherein the gene of interest encodes an insecticidal protein, a fungicidal protein, a bactericidal protein, or a viricidal protein.
12. The construct of any one of embodiments 1-11, wherein the gene of interest encodes an insecticidal protein derived from Bacillus thuringiensis.
13. The construct of any one of embodiments 1-12, wherein the gene of interest encodes a Vip or Cry protein derived from Bacillus thuringiensis.
14. The construct of any one of embodiments 1-13, wherein increased expression of the gene of interest in the host plant improves a growing parameter, a production parameter, or a biophysical parameter of the host plant.
15. The construct of any one of embodiments 1-14, wherein the genetic cargo comprises genes for the production of a biomolecule.
16. The construct of any one of embodiments 1-15, wherein the genetic cargo comprises genes for the production of a biomolecule, wherein the biomolecule is a protein, lipid, carbohydrate, or nucleic acid.
17. The construct of any one of embodiments 1-16, wherein increased expression of the gene of interest in the host plant improves the host plant response to stress.
18. The construct of any one of embodiments 1-17, wherein the genetic cargo comprises sequences and/or genes for the improved expression of the gene of interest.
19. The construct of any one of embodiments 1-18, wherein the siRNA sequence is integrated into the host factor sequence.
20. The construct of any one of embodiments 1-19, wherein the siRNA sequence is integrated into an intron in the host factor sequence.
21. The construct of any one of embodiments 1-20, wherein the siRNA sequence is integrated into an exon in the host factor sequence.
22. The construct of any one of embodiments 1-21, wherein the genetic construct comprises a promoter, and wherein the siRNA sequence is integrated into the promoter region.
23. The construct of any one of embodiments 1-22, wherein the siRNA sequence is a sequence endogenous to the host plant cell.
24. The construct of any one of embodiments 1-23, wherein the siRNA sequence is a sequence from a transposable element known to be silenced within the plant cell.
25. The construct of any one of embodiments 1-24, wherein the construct comprises a promoter.
26. The construct of any one of embodiments 1-25, wherein the construct comprises a promoter, and wherein the promoter is an inducible promoter, a constitutive promoter, a tissue-specific promoter, or a tissue-preferred promoter.
27. The construct of any one of embodiments 1-26, wherein the genetic cargo comprises a promoter.
28. The construct of any one of embodiments 1-27, wherein the construct comprises a promoter that acts on both the host factor sequence and the genetic cargo.
29. The construct of any one of embodiments 1-28, wherein the construct comprises independent promoters that act on each of the host factor sequence and the genetic cargo.
30. The construct of any one of embodiments 1-29, wherein the construct comprises independent promoters that act on each of the host factor sequence and the genetic cargo, and wherein the promoters are differentially induced.
31. The construct of any one of embodiments 1-30, wherein the construct comprises a promoter, and wherein the promoter is endogenous to the host plant cell.
32. The construct of any one of embodiments 1-31, wherein the construct comprises a promoter, and wherein the promoter is a transgenic or synthetic promoter.
33. A transgenic plant, plant part, or plant cell comprising the genetic construct of any one of embodiments 1-32.
34. The transgenic plant, plant part, or plant cell of embodiment 33, wherein the plant, plant part, or plant cell is an agronomic crop plant, plant part, or plant cell.
35. The transgenic plant, plant part, or plant cell of embodiment 33 or 34, wherein the plant, plant part, or plant cell is in the subfamily Papilionoideae.
36. The transgenic plant, plant part, or plant cell of any one of embodiments 33-35, wherein the plant, plant part, or plant cell is in the subfamily Papilionoideae and is in the species Glycine.
37. The transgenic plant, plant part, or plant cell of any one of embodiments 33-36, wherein the plant, plant part, or plant cell is in the family Poaceae.
38. The transgenic plant, plant part, or plant cell of any one of embodiments 33-37, wherein the plant, plant part, or plant cell is in the family Poaceae and is in the species Oryza, Zea, Triticum, or Saccharum.
39. The transgenic plant, plant part, or plant cell of any one of embodiments 33-38, wherein the plant, plant part, or plant cell is in the family Malvaceae.
40. The transgenic plant, plant part, or plant cell of any one of embodiments 33-39, wherein the plant, plant part, or plant cell is in the family Malvaceae and is in the species Gossypium.
41. The transgenic plant, plant part, or plant cell of any one of embodiments 33-40, wherein the plant, plant part, or plant cell is a horticultural plant, plant part, or plant cell.
42. A vector comprising the construct of any one of embodiments 1-32.
43. The vector of embodiment 42, wherein the vector is a viral vector, plasmid, retrotransposon-based vector, or a CRISPR-based vector.
44. The vector of embodiment 42 or 43, wherein the vector comprises a CRISPR-Cas nuclease.
45. The vector of any one of embodiments 42-44, wherein the vector comprises a CRISPR-Cas nuclease, gRNAs, and homology arms.
46. The vector of any one of embodiments 42-45, wherein the vector comprises a CRISPR-Cas nuclease, gRNAs, and homology arms, and wherein the sequence homologous to the host factor gene is integrated into one of the homology arms.
47. A transgenic plant, or part thereof, comprising a genetic construct comprising: (a) a plant host factor sequence operably linked to a genetic cargo sequence comprising a gene of interest: and (b) an siRNA sequence, wherein the siRNA sequence increases the likelihood that silencing of the genetic construct will lead to silencing of the plant host factor sequence.
48. The transgenic plant of embodiment 47, wherein the host factor sequence is a host factor gene, a fragment thereof, or a sequence homologous thereto.
49. The transgenic plant of embodiment 47 or 48, wherein the host factor is involved in host plant fecundity, survival, or gene silencing.
50. The transgenic plant of any one of embodiments 47-49, further comprising one or more host factor exon sequences at a 5′ and/or 3′ UTR with or without siRNA inclusions to direct silencing.
51. The transgenic plant of any one of embodiments 47-50, wherein the host factor is a DNA-directed RNA polymerase, a DNA methyltransferase, a dicer protein, a dicer-like protein, an argonaute protein, or an RNA-dependent RNA polymerase.
52. The transgenic plant of any one of embodiments 47-51, wherein the host factor is involved in host plant gene silencing via RNA-directed DNA methylation.
53. The transgenic plant of any one of embodiments 47-52, wherein the host factor is Argonaute 4 (AGO4), DNA-directed RNA polymerase V, or DNA methyltransferase.
54. The transgenic plant of any one of embodiments 47-53, wherein the gene of interest is a gene endogenous to the host plant cell.
55. The transgenic plant of any one of embodiments 47-54, wherein the gene of interest is a transgene.
56. The transgenic plant of any one of embodiments 47-55, wherein the gene of interest encodes a biocidal protein.
57. The transgenic plant of any one of embodiments 47-56, wherein the gene of interest encodes an insecticidal protein, a fungicidal protein, a bactericidal protein, or a viricidal protein.
58. The transgenic plant of any one of embodiments 47-57, wherein the gene of interest encodes an insecticidal protein derived from Bacillus thuringiensis.
59. The transgenic plant of any one of embodiments 47-58, wherein the gene of interest encodes a Vip or Cry protein derived from Bacillus thuringiensis.
60. The transgenic plant of any one of embodiments 47-59, wherein increased expression of the gene of interest in the host plant improves a growing parameter, a production parameter, or a biophysical parameter of the host plant.
61. The transgenic plant of any one of embodiments 47-60, wherein the genetic cargo comprises genes for the production of a biomolecule.
62. The transgenic plant of any one of embodiments 47-61, wherein the genetic cargo comprises genes for the production of a biomolecule, wherein the biomolecule is a protein, lipid, carbohydrate, or nucleic acid.
63. The transgenic plant of any one of embodiments 47-62, wherein increased expression of the gene of interest in the host plant improves the host plant response to stress.
64. The transgenic plant of any one of embodiments 47-63, wherein the genetic cargo comprises sequences and/or genes for the improved expression of the gene of interest.
65. The transgenic plant of any one of embodiments 47-64, wherein the siRNA sequence is integrated into the host factor sequence.
66. The transgenic plant of any one of embodiments 47-65, wherein the siRNA sequence is integrated into an intron in the host factor sequence.
67. The transgenic plant of any one of embodiments 47-66, wherein the siRNA sequence is integrated into an exon in the host factor sequence.
68. The transgenic plant of any one of embodiments 47-67, wherein the genetic construct comprises a promoter, and wherein the siRNA sequence is integrated into the promoter region.
69. The transgenic plant of any one of embodiments 47-68, wherein the siRNA sequence is a sequence endogenous to the host plant cell.
70. The transgenic plant of any one of embodiments 47-69, wherein the siRNA sequence is a sequence from a transposable element known to be silenced within the plant cell.
71. The transgenic plant of any one of embodiments 47-70, wherein the construct comprises a promoter.
72. The transgenic plant of any one of embodiments 47-71, wherein the construct comprises a promoter, and wherein the promoter is an inducible promoter, a constitutive promoter, a tissue-specific promoter, or a tissue-preferred promoter.
73. The transgenic plant of any one of embodiments 47-72, wherein the genetic cargo comprises a promoter.
74. The transgenic plant of any one of embodiments 47-73, wherein the construct comprises a promoter that acts on both the host factor sequence and the genetic cargo.
75. The transgenic plant of any one of embodiments 47-74, wherein the construct comprises independent promoters that act on each of the host factor sequence and the genetic cargo.
76. The transgenic plant of any one of embodiments 47-75, wherein the construct comprises independent promoters that act on each of the host factor sequence and the genetic cargo, and wherein the promoters are differentially induced.
77. The transgenic plant of any one of embodiments 47-76, wherein the construct comprises a promoter, and wherein the promoter is endogenous to the host plant cell.
78. The transgenic plant of any one of embodiments 47-77, wherein the construct comprises a promoter, and wherein the promoter is a transgenic or synthetic promoter.
79. The transgenic plant of any one of embodiments 47-78, wherein the plant, plant part, or plant cell is an agronomic crop plant, plant part, or plant cell
80. The transgenic plant of any one of embodiments 47-79, wherein the plant, plant part, or plant cell is in the subfamily Papilionoideae.
81. The transgenic plant of any one of embodiments 47-80, wherein the plant, plant part, or plant cell is in the subfamily Papilionoideae and is in the species Glycine.
82. The transgenic plant of any one of embodiments 47-81, wherein the plant, plant part, or plant cell is in the family Poaceae.
83. The transgenic plant of any one of embodiments 47-82, wherein the plant, plant part, or plant cell is in the family Poaceae and is in the species Oryza, Zea, Triticum, or Saccharum.
84. The transgenic plant of any one of embodiments 47-83, wherein the plant, plant part, or plant cell is in the family Malvaceae.
85. The transgenic plant of any one of embodiments 47-84, wherein the plant, plant part, or plant cell is in the family Malvaceae and is in the species Gossypium.
86. The transgenic plant of any one of embodiments 47-85, wherein the plant, plant part, or plant cell is a horticultural plant, plant part, or plant cell.
87. A method of delivering a genetic construct according to any one of embodiments 1-32 to a host plant cell.
88. A method of suppressing endogenous silencing of a gene of interest in a plant, plant part, or plant cell, comprising: transforming the plant, plant part, or plant cell with a genetic construct comprising: (a) (b) an siRNA sequence, wherein the siRNA sequence increases the likelihood that silencing of the genetic construct will lead to silencing of the plant host factor sequence, wherein expression of the genetic construct suppresses endogenous silencing of the gene of interest.
89. A method of increasing expression of a gene of interest in a plant, plant part, or plant cell, comprising: transforming the plant, plant part, or plant cell with a genetic construct comprising: (a) a plant host factor sequence operably linked to a genetic cargo sequence comprising a gene of interest: and (b) an siRNA sequence, wherein the siRNA sequence increases the likelihood that silencing of the genetic construct will lead to silencing of the plant host factor sequence, wherein expression of the genetic construct suppresses endogenous silencing of the gene of interest.
90. The method of embodiment 88 or 89, wherein the host factor sequence is a host factor gene, a fragment thereof, or a sequence homologous thereto.
91. The method of any one of embodiments 88-90, wherein the host factor is involved in host plant fecundity, survival, or gene silencing.
92. The method of any one of embodiments 88-91, further comprising one or more host factor exon sequences at a 5′ and/or 3′ UTR with or without siRNA inclusions to direct silencing.
93. The method of any one of embodiments 88-92, wherein the host factor is a DNA-directed RNA polymerase, a DNA methyltransferase, a dicer protein, a dicer-like protein, an argonaute protein, or an RNA-dependent RNA polymerase.
94. The method of any one of embodiments 88-93, wherein the host factor is involved in host plant gene silencing via RNA-directed DNA methylation.
95. The method of any one of embodiments 88-94, wherein the host factor is Argonaute 4 (AGO4), DNA-directed RNA polymerase V, or DNA methyltransferase.
96. The method of any one of embodiments 88-95, wherein the gene of interest is a gene endogenous to the host plant cell.
97. The method of any one of embodiments 88-96, wherein the gene of interest is a transgene.
98. The method of any one of embodiments 88-97, wherein the gene of interest encodes a biocidal protein.
99. The method of any one of embodiments 88-98, wherein the gene of interest encodes an insecticidal protein, a fungicidal protein, a bactericidal protein, or a viricidal protein.
100. The method of any one of embodiments 88-99, wherein the gene of interest encodes an insecticidal protein derived from Bacillus thuringiensis.
101. The method of any one of embodiments 88-100, wherein the gene of interest encodes a Vip or Cry protein derived from Bacillus thuringiensis.
102. The method of any one of embodiments 88-101, wherein increased expression of the gene of interest in the host plant improves a growing parameter, a production parameter, or a biophysical parameter of the host plant.
103. The method of any one of embodiments 88-102, wherein the genetic cargo comprises genes for the production of a biomolecule.
104. The method of any one of embodiments 88-103, wherein the genetic cargo comprises genes for the production of a biomolecule, wherein the biomolecule is a protein, lipid, carbohydrate, or nucleic acid.
105. The method of any one of embodiments 88-104, wherein increased expression of the gene of interest in the host plant improves the host plant response to stress.
106. The method of any one of embodiments 88-105, wherein the genetic cargo comprises sequences and/or genes for the improved expression of the gene of interest.
107. The method of any one of embodiments 88-106, wherein the siRNA sequence is integrated into the host factor sequence.
108. The method of any one of embodiments 88-107, wherein the siRNA sequence is integrated into an intron in the host factor sequence.
109. The method of any one of embodiments 88-108, wherein the siRNA sequence is integrated into an exon in the host factor sequence.
110. The method of any one of embodiments 88-109, wherein the genetic construct comprises a promoter, and wherein the siRNA sequence is integrated into the promoter region.
111. The method of any one of embodiments 88-110, wherein the siRNA sequence is a sequence endogenous to the host plant cell.
112. The method of any one of embodiments 88-111, wherein the siRNA sequence is a sequence from a transposable element known to be silenced within the plant cell.
113. The method of any one of embodiments 88-112, wherein the construct comprises a promoter.
114. The method of any one of embodiments 88-113, wherein the construct comprises a promoter, and wherein the promoter is an inducible promoter, a constitutive promoter, a tissue-specific promoter, or a tissue-preferred promoter.
115. The method of any one of embodiments 88-114, wherein the genetic cargo comprises a promoter.
116. The method of any one of embodiments 88-115, wherein the construct comprises a promoter that acts on both the host factor sequence and the genetic cargo.
117. The method of any one of embodiments 88-116, wherein the construct comprises independent promoters that act on each of the host factor sequence and the genetic cargo.
118. The method of any one of embodiments 88-117, wherein the construct comprises independent promoters that act on each of the host factor sequence and the genetic cargo, and wherein the promoters are differentially induced.
119. The method of any one of embodiments 88-118, wherein the construct comprises a promoter, and wherein the promoter is endogenous to the host plant cell.
120. The method of any one of embodiments 88-119, wherein the construct comprises a promoter, and wherein the promoter is a transgenic or synthetic promoter.
121. The method of any one of embodiments 88-120, wherein the plant, plant part, or plant cell is an agronomic crop plant, plant part, or plant cell
122. The method of any one of embodiments 88-121, wherein the plant, plant part, or plant cell is in the subfamily Papilionoideae.
123. The method of any one of embodiments 88-122, wherein the plant, plant part, or plant cell is in the subfamily Papilionoideae and is in the species Glycine.
124. The method of any one of embodiments 88-123, wherein the plant, plant part, or plant cell is in the family Poaceae.
125. The method of any one of embodiments 88-124, wherein the plant, plant part, or plant cell is in the family Poaceae and is in the species Oryza, Zea, Triticum, or Saccharum.
126. The method of any one of embodiments 88-125, wherein the plant, plant part, or plant cell is in the family Malvaceae.
127. The method of any one of embodiments 88-126, wherein the plant, plant part, or plant cell is in the family Malvaceae and is in the species Gossypium.
128. The method of any one of embodiments 88-127, wherein the plant, plant part, or plant cell is a horticultural plant, plant part, or plant cell.
129 The method of any one of embodiments 88-128, wherein the method increases expression of the target gene in the host plant cell.
130. The method of any one of embodiments 88-129, wherein the method reduces transcriptional or translational silencing of the target gene.
131. The method of any one of embodiments 88-130, wherein the method reduces methylation of the target gene.
132. The method of any one of embodiments 88-131, wherein the method leads to increased production of a target biomolecule.

1. A genetic construct comprising:
a) a plant host factor sequence operably linked to a genetic cargo sequence comprising a gene of interest: and
b) an siRNA sequence, wherein the siRNA sequence increases the likelihood that silencing of the genetic construct will lead to silencing of the plant host factor sequence.

2. The construct of claim 1, wherein the host factor sequence is a host factor gene, a fragment thereof, or a sequence homologous thereto.

3. The construct of claim 1, wherein the host factor is involved in host plant fecundity, survival, or gene silencing.

4. The construct of claim 1, further comprising one or more host factor exon sequences at a 5′ and/or 3′ UTR with or without siRNA inclusions to direct silencing.

5. The construct of claim 1, wherein the host factor is a DNA-directed RNA polymerase, a DNA methyltransferase, a dicer protein, a dicer-like protein, an argonaute protein, or an RNA-dependent RNA polymerase.

6. The construct of claim 1, wherein the host factor is involved in host plant gene silencing via RNA-directed DNA methylation.

7. The construct of claim 1, wherein the host factor is Argonaute 4 (AGO4), DNA-directed RNA polymerase V, or DNA methyltransferase.

8. The construct of claim 1, wherein the gene of interest is a gene endogenous to the host plant cell.

9. The construct of claim 1, wherein the gene of interest is a transgene.

10. The construct of claim 1, wherein the gene of interest encodes a biocidal protein.

11. The construct of claim 1, wherein the gene of interest encodes an insecticidal protein, a fungicidal protein, a bactericidal protein, or a viricidal protein.

12. The construct of claim 1, wherein the gene of interest encodes an insecticidal protein derived from Bacillus thuringiensis.

13. The construct of claim 1, wherein the gene of interest encodes a Vip or Cry protein derived from Bacillus thuringiensis.

14. The construct of claim 1, wherein increased expression of the gene of interest in the host plant improves a growing parameter, a production parameter, or a biophysical parameter of the host plant.

15. The construct of claim 1, wherein the genetic cargo comprises genes for the production of a biomolecule.

16. The construct of claim 1, wherein the genetic cargo comprises genes for the production of a biomolecule, wherein the biomolecule is a protein, lipid, carbohydrate, or nucleic acid.

17. The construct of claim 1, wherein increased expression of the gene of interest in the host plant improves the host plant response to stress.

18. The construct of claim 1, wherein the genetic cargo comprises sequences and/or genes for the improved expression of the gene of interest.

19. The construct of claim 1, wherein the siRNA sequence is integrated into the host factor sequence.

20. The construct of claim 1, wherein the siRNA sequence is integrated into an intron in the host factor sequence.

21. The construct of claim 1, wherein the siRNA sequence is integrated into an exon in the host factor sequence.

22. The construct of claim 1, wherein the genetic construct comprises a promoter, and wherein the siRNA sequence is integrated into the promoter region.

23. The construct of claim 1, wherein the siRNA sequence is a sequence endogenous to the host plant cell.

24. The construct of claim 1, wherein the siRNA sequence is a sequence from a transposable element known to be silenced within the plant cell.

25. The construct of claim 1, wherein the construct comprises a promoter.

26. The construct of claim 1, wherein the construct comprises a promoter, and wherein the promoter is an inducible promoter, a constitutive promoter, a tissue-specific promoter, or a tissue-preferred promoter.

27. The construct of claim 1, wherein the genetic cargo comprises a promoter.

28. The construct of claim 1, wherein the construct comprises a promoter that acts on both the host factor sequence and the genetic cargo.

29. The construct of claim 1, wherein the construct comprises independent promoters that act on each of the host factor sequence and the genetic cargo.

30. The construct of claim 1, wherein the construct comprises independent promoters that act on each of the host factor sequence and the genetic cargo, and wherein the promoters are differentially induced.

31. The construct of claim 1, wherein the construct comprises a promoter, and wherein the promoter is endogenous to the host plant cell.

32. The construct of claim 1, wherein the construct comprises a promoter, and wherein the promoter is a transgenic or synthetic promoter.

33. A transgenic plant, plant part, or plant cell comprising the genetic construct of any one of claims 1-32.

34. The transgenic plant, plant part, or plant cell of claim 33, wherein the plant, plant part, or plant cell is an agronomic crop plant, plant part, or plant cell.

35. The transgenic plant, plant part, or plant cell of claim 33, wherein the plant, plant part, or plant cell is in the subfamily Papilionoideae.

36. The transgenic plant, plant part, or plant cell of claim 33, wherein the plant, plant part, or plant cell is in the subfamily Papilionoideae and is in the species Glycine.

37. The transgenic plant, plant part, or plant cell of claim 33, wherein the plant, plant part, or plant cell is in the family Poaceae.

38. The transgenic plant, plant part, or plant cell of claim 33, wherein the plant, plant part, or plant cell is in the family Poaceae and is in the species Oryza, Zea, Triticum, or Saccharum.

39. The transgenic plant, plant part, or plant cell of claim 33, wherein the plant, plant part, or plant cell is in the family Malvaceae.

40. The transgenic plant, plant part, or plant cell of claim 33, wherein the plant, plant part, or plant cell is in the family Malvaceae and is in the species Gossypium.

41. The transgenic plant, plant part, or plant cell of claim 33, wherein the plant, plant part, or plant cell is a horticultural plant, plant part, or plant cell.

42. A vector comprising the construct of any one of claims 1-32.

43. The vector of claim 42, wherein the vector is a viral vector, plasmid, retrotransposon-based vector, or a CRISPR-based vector.

44. The vector of claim 42, wherein the vector comprises a CRISPR-Cas nuclease.

45. The vector of claim 42, wherein the vector comprises a CRISPR-Cas nuclease, gRNAs, and homology arms.

46. The vector of claim 42, wherein the vector comprises a CRISPR-Cas nuclease, gRNAs, and homology arms, and wherein the sequence homologous to the host factor gene is integrated into one of the homology arms.

47. A transgenic plant, or part thereof, comprising a genetic construct comprising:
a) a plant host factor sequence operably linked to a genetic cargo sequence comprising a gene of interest; and
b) an siRNA sequence, wherein the siRNA sequence increases the likelihood that silencing of the genetic construct will lead to silencing of the plant host factor sequence.

48. The transgenic plant of claim 47, wherein the host factor sequence is a host factor gene, a fragment thereof, or a sequence homologous thereto.

49. The transgenic plant of claim 47, wherein the host factor is involved in host plant fecundity, survival, or gene silencing.

50. The transgenic plant of claim 47, further comprising one or more host factor exon sequences at a 5′ and/or 3′ UTR with or without siRNA inclusions to direct silencing.

51. The transgenic plant of claim 47, wherein the host factor is a DNA-directed RNA polymerase, a DNA methyltransferase, a dicer protein, a dicer-like protein, an argonaute protein, or an RNA-dependent RNA polymerase.

52. The transgenic plant of claim 47, wherein the host factor is involved in host plant gene silencing via RNA-directed DNA methylation.

53. The transgenic plant of claim 47, wherein the host factor is Argonaute 4 (AGO4), DNA-directed RNA polymerase V, or DNA methyltransferase.

54. The transgenic plant of claim 47, wherein the gene of interest is a gene endogenous to the host plant cell.

55. The transgenic plant of claim 47, wherein the gene of interest is a transgene.

56. The transgenic plant of claim 47, wherein the gene of interest encodes a biocidal protein.

57. The transgenic plant of claim 47, wherein the gene of interest encodes an insecticidal protein, a fungicidal protein, a bactericidal protein, or a viricidal protein.

58. The transgenic plant of claim 47, wherein the gene of interest encodes an insecticidal protein derived from Bacillus thuringiensis.

59. The transgenic plant of claim 47, wherein the gene of interest encodes a Vip or Cry protein derived from Bacillus thuringiensis.

60. The transgenic plant of claim 47, wherein increased expression of the gene of interest in the host plant improves a growing parameter, a production parameter, or a biophysical parameter of the host plant.

61. The transgenic plant of claim 47, wherein the genetic cargo comprises genes for the production of a biomolecule.

62. The transgenic plant of claim 47, wherein the genetic cargo comprises genes for the production of a biomolecule, wherein the biomolecule is a protein, lipid, carbohydrate, or nucleic acid.

63. The transgenic plant of claim 47, wherein increased expression of the gene of interest in the host plant improves the host plant response to stress.

64. The transgenic plant of claim 47, wherein the genetic cargo comprises sequences and/or genes for the improved expression of the gene of interest.

65. The transgenic plant of claim 47, wherein the siRNA sequence is integrated into the host factor sequence.

66. The transgenic plant of claim 47, wherein the siRNA sequence is integrated into an intron in the host factor sequence.

67. The transgenic plant of claim 47, wherein the siRNA sequence is integrated into an exon in the host factor sequence.

68. The transgenic plant of claim 47, wherein the genetic construct comprises a promoter, and wherein the siRNA sequence is integrated into the promoter region.

69. The transgenic plant of claim 47, wherein the siRNA sequence is a sequence endogenous to the host plant cell.

70. The transgenic plant of claim 47, wherein the siRNA sequence is a sequence from a transposable element known to be silenced within the plant cell.

71. The transgenic plant of claim 47, wherein the construct comprises a promoter.

72. The transgenic plant of claim 47, wherein the construct comprises a promoter, and wherein the promoter is an inducible promoter, a constitutive promoter, a tissue-specific promoter, or a tissue-preferred promoter.

73. The transgenic plant of claim 47, wherein the genetic cargo comprises a promoter.

74. The transgenic plant of claim 47, wherein the construct comprises a promoter that acts on both the host factor sequence and the genetic cargo.

75. The transgenic plant of claim 47, wherein the construct comprises independent promoters that act on each of the host factor sequence and the genetic cargo.

76. The transgenic plant of claim 47, wherein the construct comprises independent promoters that act on each of the host factor sequence and the genetic cargo, and wherein the promoters are differentially induced.

77. The transgenic plant of claim 47, wherein the construct comprises a promoter, and wherein the promoter is endogenous to the host plant cell.

78. The transgenic plant of claim 47, wherein the construct comprises a promoter, and wherein the promoter is a transgenic or synthetic promoter.

79. The transgenic plant of claim 47, wherein the plant, plant part, or plant cell is an agronomic crop plant, plant part, or plant cell

80. The transgenic plant of claim 47, wherein the plant, plant part, or plant cell is in the subfamily Papilionoideae.

81. The transgenic plant of claim 47, wherein the plant, plant part, or plant cell is in the subfamily Papilionoideae and is in the species Glycine.

82. The transgenic plant of claim 47, wherein the plant, plant part, or plant cell is in the family Poaceae.

83. The transgenic plant of claim 47, wherein the plant, plant part, or plant cell is in the family Poaceae and is in the species Oryza, Zea, Triticum, or Saccharum.

84. The transgenic plant of claim 47, wherein the plant, plant part, or plant cell is in the family Malvaceae.

85. The transgenic plant of claim 47, wherein the plant, plant part, or plant cell is in the family Malvaceae and is in the species Gossypium.

86. The transgenic plant of claim 47, wherein the plant, plant part, or plant cell is a horticultural plant, plant part, or plant cell.

87. A method of delivering a genetic construct according to any one of claims 1-32 to a host plant cell.

88. A method of suppressing endogenous silencing of a gene of interest in a plant, plant part, or plant cell, comprising:
transforming the plant, plant part, or plant cell with a genetic construct comprising:
a) a plant host factor sequence operably linked to a genetic cargo sequence comprising a gene of interest: and
b) an siRNA sequence, wherein the siRNA sequence increases the likelihood that silencing of the genetic construct will lead to silencing of the plant host factor sequence,
wherein expression of the genetic construct suppresses endogenous silencing of the gene of interest.

89. A method of increasing expression of a gene of interest in a plant, plant part, or plant cell, comprising:
transforming the plant, plant part, or plant cell with a genetic construct comprising:
a) a plant host factor sequence operably linked to a genetic cargo sequence comprising a gene of interest; and
b) an siRNA sequence, wherein the siRNA sequence increases the likelihood that silencing of the genetic construct will lead to silencing of the plant host factor sequence,
wherein expression of the genetic construct suppresses endogenous silencing of the gene of interest.

90. The method of claim 88 or 89, wherein the host factor sequence is a host factor gene, a fragment thereof, or a sequence homologous thereto.

91. The method of claim 88 or 89, wherein the host factor is involved in host plant fecundity, survival, or gene silencing.

92. The method of claim 88 or 89, further comprising one or more host factor exon sequences at a 5′ and/or 3′ UTR with or without siRNA inclusions to direct silencing.

93. The method of claim 88 or 89, wherein the host factor is a DNA-directed RNA polymerase, a DNA methyltransferase, a dicer protein, a dicer-like protein, an argonaute protein, or an RNA-dependent RNA polymerase.

94. The method of claim 88 or 89, wherein the host factor is involved in host plant gene silencing via RNA-directed DNA methylation.

95. The method of claim 88 or 89, wherein the host factor is Argonaute 4 (AGO4), DNA-directed RNA polymerase V, or DNA methyltransferase.

96. The method of claim 88 or 89, wherein the gene of interest is a gene endogenous to the host plant cell.

97. The method of claim 88 or 89, wherein the gene of interest is a transgene.

98. The method of claim 88 or 89, wherein the gene of interest encodes a biocidal protein.

99. The method of claim 88 or 89, wherein the gene of interest encodes an insecticidal protein, a fungicidal protein, a bactericidal protein, or a viricidal protein.

100. The method of claim 88 or 89, wherein the gene of interest encodes an insecticidal protein derived from Bacillus thuringiensis.

101. The method of claim 88 or 89, wherein the gene of interest encodes a Vip or Cry protein derived from Bacillus thuringiensis.

102. The method of claim 88 or 89, wherein increased expression of the gene of interest in the host plant improves a growing parameter, a production parameter, or a biophysical parameter of the host plant.

103. The method of claim 88 or 89, wherein the genetic cargo comprises genes for the production of a biomolecule.

104. The method of claim 88 or 89, wherein the genetic cargo comprises genes for the production of a biomolecule, wherein the biomolecule is a protein, lipid, carbohydrate, or nucleic acid.

105. The method of claim 88 or 89, wherein increased expression of the gene of interest in the host plant improves the host plant response to stress.

106. The method of claim 88 or 89, wherein the genetic cargo comprises sequences and/or genes for the improved expression of the gene of interest.

107. The method of claim 88 or 89, wherein the siRNA sequence is integrated into the host factor sequence.

108. The method of claim 88 or 89, wherein the siRNA sequence is integrated into an intron in the host factor sequence.

109. The method of claim 88 or 89, wherein the siRNA sequence is integrated into an exon in the host factor sequence.

110. The method of claim 88 or 89, wherein the genetic construct comprises a promoter, and wherein the siRNA sequence is integrated into the promoter region.

111. The method of claim 88 or 89, wherein the siRNA sequence is a sequence endogenous to the host plant cell.

112. The method of claim 88 or 89, wherein the siRNA sequence is a sequence from a transposable element known to be silenced within the plant cell.

113. The method of claim 88 or 89, wherein the construct comprises a promoter.

114. The method of claim 88 or 89, wherein the construct comprises a promoter, and wherein the promoter is an inducible promoter, a constitutive promoter, a tissue-specific promoter, or a tissue-preferred promoter.

115. The method of claim 88 or 89, wherein the genetic cargo comprises a promoter.

116. The method of claim 88 or 89, wherein the construct comprises a promoter that acts on both the host factor sequence and the genetic cargo.

117. The method of claim 88 or 89, wherein the construct comprises independent promoters that act on each of the host factor sequence and the genetic cargo.

118. The method of claim 88 or 89, wherein the construct comprises independent promoters that act on each of the host factor sequence and the genetic cargo, and wherein the promoters are differentially induced.

119. The method of claim 88 or 89, wherein the construct comprises a promoter, and wherein the promoter is endogenous to the host plant cell.

120. The method of claim 88 or 89, wherein the construct comprises a promoter, and wherein the promoter is a transgenic or synthetic promoter.

121. The method of claim 88 or 89, wherein the plant, plant part, or plant cell is an agronomic crop plant, plant part, or plant cell

122. The method of claim 88 or 89, wherein the plant, plant part, or plant cell is in the subfamily Papilionoideae.

123. The method of claim 88 or 89, wherein the plant, plant part, or plant cell is in the subfamily Papilionoideae and is in the species Glycine.

124. The method of claim 88 or 89, wherein the plant, plant part, or plant cell is in the family Poaceae.

125. The method of claim 88 or 89, wherein the plant, plant part, or plant cell is in the family Poaceae and is in the species Oryza, Zea, Triticum, or Saccharum.

126. The method of claim 88 or 89, wherein the plant, plant part, or plant cell is in the family Malvaceae.

127. The method of claim 88 or 89, wherein the plant, plant part, or plant cell is in the family Malvaceae and is in the species Gossypium.

128. The method of claim 88 or 89, wherein the plant, plant part, or plant cell is a horticultural plant, plant part, or plant cell.

129. The method of claim 88 or 89, wherein the method increases expression of the target gene in the host plant cell.

130. The method of claim 88 or 89, wherein the method reduces transcriptional or translational silencing of the target gene.

131. The method of claim 88 or 89, wherein the method reduces methylation of the target gene.

132. The method of claim 88 or 89, wherein the method leads to increased production of a target biomolecule.
